(19) United States
(12) Patent Application Publication
(10) Pub. No.: US 2005/0214263 A1
(43) Pub. Date: Sep. 29, 2005

(54) Methods and means for gene silencing in plants

(76) Inventors: Fabian E. Vaistij, York (GB); Olivier Voinnet, Strasbourg (FR); David Charles Baulcombe, Norwich (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/498,660

(22) PCT Filed: Dec. 5, 2002
(86) PCT No.: PCT/GB02/05505

§ 371 Date: Jan. 14, 2005

(30) Foreign Application Priority Data
Dec. 12, 2001 (GB) 01297548
Jan. 3, 2002 (GB) 02000982

(51) Int. Cl. A61K 48/00

(52) U.S. Cl. 424/93.21; 514/44

(57) ABSTRACT

Provided are methods of silencing a target gene in an organism, which methods comprises the steps of: (a) providing a recombinant DNA construct including an expression cassette comprising: (i) a promoter, operably linked to, (ii) a chimeric nucleotide sequence encoding all or part of the target gene and a transgene, (b) transforming the organism with said DNA construct such that the expression cassette is inserted into the genome, and (c) initiating post transcriptional gene silencing (PTGS) of said transgene in said organism, whereby initiation of PTGS of the transgene causes silencing of the target gene in the organism. The methods are based on a phenomenon termed “spreading” whereby PTGS of the transgene can be used to spread in trans to silence, for example, endogenous target genes in the same genetic background as the chimeric gene to give consistent, maintained, silencing thereof. Also provided are related materials (e.g. recombinant constructs) and uses and methods based on these.

### TECHNICAL FIELD

The present invention relates generally to gene silencing methods and materials employing recombinant gene constructs.

### BACKGROUND ART

Being able to silence target genes in organisms such as plants is of great interest as a means of modifying the phenotype of those organisms.

RNA silencing is a nucleotide sequence-specific process of RNA degradation in higher plants (post-transcriptional gene silencing, PTGS), animals (RNA interference, RNAi) and fungi (quelling) as well as in unicellular eukaryotic algae (Carthew, 2001; Matzke et al., 2001; Waterhouse et al., 2001). In higher plants a natural role of RNA silencing is to protect against viruses (Al-Kaff et al., 1998; Covey et al., 1997; Hamilton and Baulcombe, 1999; Ratcliff et al., 1997). A role in genome protection is also likely because there is enhanced transposon mobility in RNA silencing-defective mutants of C. reinhardtii, C. elegans and transposition is suppressed by RNA silencing in D. melagonaster (Jensen et al., 1999; Ketting and Plasterk, 2000; Wu-Scharf et al., 2000).

Double-stranded RNA (dsRNA) is a potent activator of RNA silencing (Fire et al., 1998). Consequently RNA silencing is activated by viral RNAs that replicate via double-stranded intermediates and by transgenes with inverted repeat (IR) structures that could be transcribed into dsRNA (Chuang and Meyerowitz, 2000; Smith et al., 2000; Waterhouse et al., 2001). Single-copy transgenes without IR structures can also activate RNA silencing (Elmayan and Vaucheret, 1996). In these cases it is unlikely that dsRNA would be produced by direct transcription and it is thought that single-stranded RNAs (ssRNAs) are converted into dsRNAs by an RNA-dependent RNA polymerase (RdRP) (Lindbo et al., 1993). In support of this hypothesis, putative RdRPs encoded by the SDE1/SGS2, QDE1 and EGO1 loci are required for RNA silencing in A. thaliana, N. crassa and C. elegans, respectively (Cogoni and Macino, 2000; Dalmay et al., 2000b; Mourrain et al., 2000; Smardon et al., 2000).

Biochemical analyses of RNA silencing in D. melagonaster have shown that an RNaseIII (DICER) cleaves the dsRNAs into 21-25 nucleotide RNAs short interfering RNAs (siRNAs) that associate with a second RNase in an ‘RNA-induced silencing complex’ (RISC) (Bernstein et al., 2001; Hammond et al., 2000). RISC cleaves target single-stranded RNAs at a site that is complementary to the (antisense) siRNA. Thus, the role of the siRNAs is to provide sequence-specificity to RNA silencing (Elbashir et al., 2001). Plant DICER and RISC have not yet been identified. However, siRNAs are present in plant RNA silencing systems suggesting that mechanisms are conserved across kingdoms (Hamilton and Baulcombe, 1999).

In plants, viruses carrying portions of host genes can initiate silencing of the corresponding RNA through a process known as virus-induced gene silencing (VIGS) (Kumagai et al., 1995; Ruiz et al., 1998). In some instances, when the target sequence is from a transgene, the viral RNA is eventually eliminated by the silencing process. However, even in the absence of the virus, the transgene remains silenced. VIGS of transgenes is associated with sequence-specific methylation of transgene DNA (Jones et al., 1999). This process, termed RNA-directed DNA methylation (RdDM), is a consequence of dsRNA or siRNAs interacting directly with the target DNA (Wassenegger, 2000).

To account for the transition from virus-dependence to virus-independence it has been proposed that there are different phases of the RNA silencing mechanism referred to as initiation and maintenance (Ruiz et al., 1998). Consistent with this idea it was shown in Arabidopsis that the two phases could be differentiated by mutation analysis and by the methylation of a GFP transgene. In wild-type plants there is both initiation and maintenance of GFP silencing and RdDM of the GFP transgene. In contrast in sde1/sgs2 and sde3 mutants there is initiation but not maintenance and the transgene is not methylated (Dalmay et al., 2001).

However, a virus-free maintenance phase has only been observed when transgenes were targeted (Jones et al., 1999; Lindbo et al., 1993; Ruiz et al., 1998). VIGS of two different endogenous genes was not maintained in the absence of the virus, was not dependent on SDE1/SGS2 and SDE3 and did not lead to RdDM of the corresponding DNA (Dalmay et al., 2001; Jones et al., 1999; Ruiz et al., 1998; Thomas et al., 2001).

Again considering transgenes, the transition from initiation to maintenance is also observed when RNA silencing of the transgenes is triggered by delivery of ectopic DNA via Agrobacterium infiltration, by bombardment or in grafting experiments (Palauqui and Balzergue, 1999; Palauqui et al., 1997; Voinnet and Baulcombe, 1997; Voinnet et al., 1998). In these systems, localized initiation of RNA silencing triggers systemic silencing throughout the plant. However, at least following Agrobacterium infiltration or bombardment, systemic silencing is maintained even if the region in which silencing is initiated is removed.

Lipardi et al (2001) Cell Vol 107: 297-307 discuss a role for siRNAs as primers to transform mRNA into dsRNA.

Sijen et al (2001) Cell Vol 107: 1-20 discuss a role for RNA amplification in dsRNA-triggered gene silencing.

It is apparent from the forgoing that novel methods or materials for effectively silencing a target gene, such as an endogenous gene, within an organism, would represent a useful contribution to the art.

### DISCLOSURE OF THE INVENTION

The present inventors provide herein novel but powerful systems which have utility inter alia for silencing of endogenous genes. The inventors closely investigated the maintenance phase of RNA silencing and its association with spreading of both targeting and DNA methylation (as used herein, the term “spreading” is used to describe a molecular process rather than the systemic movement of a silencing signal). As a result of spreading all parts of a targeted transcript are targets of RNA silencing even if the dsRNA initiator sequence corresponds to only a fragment of it. The inventors have demonstrated inter alia that spreading and maintenance are closely associated, both seemingly being dependent on a putative RdRP. They have also demonstrated that spreading requires transcription of the target RNA. Combined these data support a model of RNA silencing in which spreading of targeting and maintenance involve production of dsRNA by an RdRP using the target sense RNA as a template.

It was known that even when a systemic gene silencing initiator is from only a part of the target GFP sequence, the maintenance phase of RNA silencing is associated with methylation of the entire transcribed region of the transgene (Jones et al., 1999; Thomas et al., 2001). Similarly, in tissues exhibiting systemic silencing of GFP, all parts of the transgene transcript were targets of RNA silencing, irrespective of whether the initiator sequence was a 5′ or 3′ fragment of the transcribed sequence (Voinnet et al., 1998). In VIGS it was known that DNA methylation spreads beyond the initiator sequence (Jones et al., 1999).

The inventors herein provide methods and materials utilising spreading in trans which in preferred embodiments may be used to provide consistent, maintained, silencing of endogenous genes, optionally in a conditional matter. No such spreading-based systems were disclosed in the prior art, and they have utility inter alia for functional genomics. For example the spreading-based system facilitates gene-function studies e.g. in A. thaliana.

Results shown herein demonstrate that it is possible to use spreading as a technology for silencing endogenes e.g. using a viral amplicon as inducer of silencing of transgene-endogene chimeras. Spreading can take place either from only one direction (3′ to 5′) or in both directions. The length of the homologous sequences can be relatively short.

Generally speaking the systems of the present invention for silencing target sequences are based on two elements (i) an initiator element which can serve to initiate gene silencing in an organism against an appropriate sequence, such as a transgene sequence, and (ii) a receptor element which includes an element which can be silenced by the initiator construct (e.g. a transgene which is identical to all or part of that initiator element) plus also a sequence identical to the intended target of silencing. The two receptor elements are present in the organism in the same genetic background e.g. introduced therein by transformation with a DNA construct.

The elements may be provided by a “triggering construct” which provides the dsRNA to initiate or trigger RNA silencing, and a “spreading construct” which carries a chimeric gene composed of a portion of the triggering construct plus sequence from an endogenous cDNA. Such a construct provides the transgenic RNA where spreading can occur, leading ultimately to silencing of the endogenous gene in the organism.

Thus in one aspect there is provided a method of silencing a target gene in an organism, which method comprises the steps of:
(a) providing a DNA construct including an expression cassette comprising (i) a promoter, operably linked to (ii) a chimeric nucleotide sequence encoding all or part of the target gene and a transgene,
(b) transforming the organism with said DNA construct such that the expression cassette is inserted into the genome, and
(c) initiating PTGS of said transgene in said organism, whereby initiation of PTGS of the transgene causes silencing of the target gene in an organism.

These steps (a)\(b) and (c) may be performed in any order i.e. the PTGS of the transgene may be initiated or extant in the organism prior to provision, introduction, or transformation of the chimeric nucleotide sequence.

“Silencing” is a term generally used to refer to suppression of expression of a gene (generally by PTGS). The degree of reduction may be so as to totally abolish production of the encoded gene product, but may also be such that the abolition of expression is not complete, with some small degree of expression remaining. The term should not therefore be taken to require complete “silencing” of expression. It is used herein where convenient because those skilled in the art well understand this. In preferred embodiments the silencing will be maintained even if the initiator of the PTGS of the transgene is removed.

“Gene” used broadly coding sequence in the DNA genome of an organism which is, or may be, expressed via transcription to mRNA and translation to a protein according to well established principles. It will generally be an endogenous gene of the organism. Preferred target genes are discussed below.

The “transgene” is foreign (non-native) to the organism, which is to say that it does not occur naturally in the organism's genome.

The “initiation” of silencing of the transgene may be by a variety of methods which are discussed in more detail hereinafter. Generally it may comprise the step of introducing into the organism a further nucleic acid construct which includes sequence corresponding to the transgene sequence such as to initiate PTGS of the transgene. As stated above, the (silenced) transgene may already be present in the organism prior to the introduction of chimeric sequence.

Where the chimeric sequence is under the control of an inducible promoter, the method may further include the step of causing or permitting transcription from the promoter such as to produce an mRNA transcript of the expression cassette.

Some embodiments and further aspects of the invention will now be described in more detail:

DNA Construct

Nucleic acid constructs according to the present invention will be recombinant and may be provided isolated and/or purified, in substantially pure or homogeneous form, or free or substantially free of other nucleic acid. The term “isolated” encompasses all these possibilities.

Since nucleic acid may be double stranded, where nucleic acid (or nucleotide sequence) of the invention is referred to herein, use of the complement of that nucleic acid (or nucleotide sequence) will also be embraced by the invention. The ‘complement’ in each case is the same length as the reference, but is 100% complementary thereto whereby by each nucleotide is base paired to its counterpart i.e. G to C, and A to T or U.

Generally speaking, in the light of the present disclosure, those skilled in the art will be able to construct vectors according to the present invention. Such vectors may include, in addition to the promoter, a suitable terminator or other regulatory sequence such as to define an expression cassette comprising the chimeric sequence.

For further details see, for example, Molecular Cloning: a Laboratory Manual: 2nd edition, Sambrook et al, 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second Edition, Ausubel et al. eds., John Wiley & Sons, 1992. Specific procedures and vectors previously used with wide success upon plants are described by Bevan, Nucl. Acids Res. (1984) 12, 8711-8721), and Guerineau and Mullineaux, (1993) Plant transformation and expression vectors. In: Plant Molecular Biology Labfax (Croy RRD ed) Oxford, BIOS Scientific Publishers, pp 121-148.

Thus an additional optional feature of a construct used in accordance with the present invention is a transcriptional terminator. For example the transcriptional terminator from nopaline synthase (nos) gene of agrobacterium tumefaciens (Depicker, A., et al (1982), J. Mol. Appl. Genet., 1: 561-573) may be used. Other suitable transcriptional terminators will be well known to those skilled in the art.

For embodiments which are practised in plants, the expression cassette will generally be situated between border sequences and are capable of being inserted into a plant genome under appropriate conditions. Generally this may be achieved by use of so called “agro-infiltration” which uses Agrobacterium-mediated transformation. Briefly, this technique is based on the property of Agrobacterium tumafaciens to transfer a portion of its DNA (“T-DNA”) into a host cell where it may become integrated into nuclear DNA. The T-DNA is defined by left and right border sequences which are around 25 nucleotides in length. In the present invention the border sequences are included around the transfer nucleotide sequence (the T-DNA) with the whole vector being introduced into the plant by agro-infiltration, optionally in the form of a binary-transformation vector. Thus the construct may include border sequences which permit the transfer of the transfer nucleotide sequence into a plant cell nucleus. Methods are described in more detail in the Examples hereinafter.

Aspects of the present invention include the isolated recombinant DNA construct described above. Also provided is a composition comprising a plurality of said constructs, each including a separate target gene (preferably from the same organism). Also provided are kits comprising one or more said constructs, and their use in the methods described herein.

Promoter

By “promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. Nucleic acid operably linked to a promoter is “under transcriptional initiation regulation” of the promoter.

In preferred embodiments the promoter may be inducible. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

In preferred embodiments, both “trigger” and “spreading” constructs are placed under individual inducible promoters such as to permit the triggering of silencing at different times and conditions.

Suitable plant active promoters will be well known to those skilled in the art. Preferred promoters may include the 35S promoter of cauliflower mosaic virus or the nopaline synthase promoter of Agrobacterium tumefaciens (Sanders, P. R., et al (1987), Nucleic Acids Res., 15: 1543-1558). These promoters are expressed in many, if not all, cell types of many plants. If the target gene is to be silenced following a defined external stimulus the construct may incorporate a promoter that is be activated specifically by that stimulus. Promoters that are both tissue specific and inducible by specific stimuli may be used. Suitable promoters may include the maize glutathione-S-transferase isoform II (GST-II-27) gene promoter which is activated in response to application of exogenous safener (WO93/01294, ICI Ltd). Another suitable (preferred) promoter may be the DEX promoter (Plant Journal (1997) 11: 605-612).

Chimeric Sequence

The chimeric sequence will include all or part of the target gene and the transgene.

In preferred embodiments the transgene is GFP or GUS, although (as shown in the examples) it could even be a viral fragment or an exogenous sequence inserted into the viral genome, such as GFP in PVX/GFP amplicon.

The part of the target gene will include more than just the promoter of the target gene, and will preferably include at least the initiating ATG codon of the target gene. It may optionally include all or part of the terminator.

The sequences need not run intact contiguously. For example the target gene may be inserted within the transgene, as in the Examples below. Provided they are ultimately present in the same genetic background, they need not form part of a single ORF.

It should be stressed that the complete sequence corresponding to the gene coding sequence (the “targeting” sequence) need not be used. For example fragments of sufficient length may be used. It is a routine matter for the person skilled in the art to screen fragments of various sizes and from various parts of the coding sequence to optimise the relationship between target and targeting sequence. Likewise it may be preferable that there is complete sequence identity between the targeting sequence in the vector and the target sequence in the plant, although total similarity of sequence is not essential. One or more nucleotides may differ in the targeting sequence from the target gene. Thus, a targeting sequence employed in a construct in accordance with the present invention may be a wild-type sequence (e.g. gene) selected from those available, or a substantially homologous mutant, derivative, variant or allele, by way of insertion, addition, deletion or substitution of one or more nucleotides, of such a sequence. A typical construct may include a sequence wherein the targeting sequence and the sequence within the target gene are substantially homologous, by which is meant that the sequence in question shares at least about 70%, or 80% identity, most preferably at least about 90%, 95%, 96%, 97%, 98% or 99% identity with the reference sequence. The sequence will preferably be at least 21, 22, 23, 24, or 25 nucleotides in length. It may be longer e.g. at least 200 nt or 745 nt.

Identity may be at the nucleotide sequence and/or encoded amino acid sequence level. Homology may be over the full-length of the relevant sequence shown herein (e.g. in the sequence Annex) or may be over a part of it. Identity may be determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Preferably sequence comparisons are made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA.

In addition to the target gene relationship, all these comments apply mutatis mutandis to the transgene in the chimeric (spreading) construct and its counterpart on the initiator (triggering) construct, where appropriate.

Choice of Target Sequence

Preferred target genes may include those which confer ‘unwanted’ traits in the plant and which it may therefore be desired to silence. Examples include ripening specific genes in tomato to improve processing and handling characteristics of the harvested fruit; genes involved in pollen formation so that breeders can reproducibly generate male sterile plants for the production of F1 hybrids; genes involved in lignin biosynthesis to improve the quality of paper pulp made from vegetative tissue of the plant; gene silencing of genes involved in flower pigment production to produce novel flower colours; gene silencing of genes involved in regulatory pathways controlling development or environmental responses to produce plants with novel growth habit or (for example) disease resistance; elimination of toxic secondary metabolites by gene silencing of genes required for toxin production.

A further possibility is to target a conserved sequence of a gene, e.g. a sequence that is characteristic of one or more genes in one or more pathogens against which resistance is desired, such as a regulatory sequence. Thus a construct may target a conserved sequence within a target gene group such as to down-regulate expression of one or more members of a target gene group. More than one targeting sequence may be included.

Choice of Transgene Silencing Method

The initial silencing step may be achieved by any conventional method appropriate to the organism in question. This then spreads in trans to the target gene.

For instance in plants it could be by silencing of the transgene by any of:
(i) VIGS—as discussed in relation to background art, viruses carrying portions of host genes (in this case transgenes) can initiate silencing of the corresponding RNA (Kumagai et al., 1995; Ruiz et al., 1998) which remains silenced even in the absence of the virus (Jones et al., 1999).

Thus in these embodiments of the present invention, initiation of PTGS of said transgene in the organism may be achieved by introducing a virus (or sequence derived therefrom) including all or part of the transgene sequence.

Preferred VIGS vectors include those based on PVX, TRV, TMV and geminiviruses (Kumagai et al., 1995; Kjemtrup et al., 1998; Ruiz et al., 1998; Peele et al., 2001; Ratcliff et al., 2001)
(ii) Transgene hairpin—Silencing, including silencing of endogenous genes, can be initiated by methods well known to those skilled in the art e.g. analogous to those described in Chuang and Meyerowitz, 2000; Smith et al., 2000; or Wesley et al., 2001. The use of such Inverted Repeats (IR) may be preferable where it is desired not to introduce replicating virus-derived material into the organism. The use of IR-based spreading systems as described herein is particularly useful for high-throughput genomic analysis.
(iii) Transgene Silencing—PTGS induced by transgenes, even in single copy, is discussed by H. Vaucheret, et al., Plant J. 16, 651-659 (1998). This is preferably provided by use of plants or lines in which ‘resident’ PTGS against transgene is already extant, and into which the chimeric DNA construct may be introduced.

In a further embodiment there is provided a method of silencing a target gene in an organism, which method comprises the steps of:
(a) providing the organism which has been transformed with a transgene which has been silenced with PTGS,
(b) transforming said organism with a DNA construct including an expression cassette comprising (i) a promoter, operably linked to (ii) a chimeric nucleotide sequence encoding all are part of the target gene and a transgene,
(iv) Systemically induced transgene silencing—in some examples of PTGS, silencing is initiated in a localised region of the plant. A signal molecule is produced at the site of initiation and mediates systemic spread of silencing to other tissues of the plant (O. Voinnet and, D. C. Baulcombe, Nature 389, 553 (1997); J.-C. Palauqui, and S. Balzergue, Curr. Biol. 9, 59 (1999))
(v) Cytoplasmically replicating constructs—silencing constructs are disclosed e.g. in WO95/34668 (Biosource). Preferred systems are those based on so-called ‘amplicons’—see Angell & Baulcombe (1997) The EMBO Journal 16, 12:3675-3684 or WO98/36083 of Plant Bioscience Limited. ‘Amplicons’, as described in WO98/36083, comprise a promoter operably linked to a viral replicase, or a promoter sequence operably linked to DNA for transcription in a plant cell of an RNA molecule that includes plant virus sequences (i.e. cis elements such as one or more sub-genomic promoters, and trans elements such as a replicase) that confer on the RNA molecule the ability to replicate in the cytoplasm of a plant cell following transcription. The transcripts replicate as if they are viral RNAs, and comprise a targeting sequence corresponding to the gene of interest (‘the target gene’). Other sequence from the viral RNA may be omitted to give a minimal amplicon. It should be stressed that the amplicon targeting gene is directed towards the transgene i.e. a transgene-targeting sequence. They may be introduced as stable transgenes into the genome of the same plant by transformation and/or crossing. Alternatively they may be introduced by agroinfiltration for transient expression.

Thus in one embodiment the invention provides a method of silencing a target gene in an organism, which method comprises the steps of:
(a) providing a first DNA construct including an expression cassette comprising (i) a promoter, operably linked to (ii) a chimeric nucleotide sequence encoding all are part of the target gene and a transgene,
(b) providing a second DNA construct including an expression cassette comprising (i) a promoter, operably linked to (ii) DNA for transcription in a plant cell of an RNA molecule that includes (I) plant virus sequences that confer on the RNA molecule the ability to replicate in the cytoplasm of a plant cell following transcription (II) a targeting sequence corresponding to the transgene,
(c) transforming the organism with said DNA constructs such that the expression cassettes are inserted into the genome, and optionally (e.g. where the promoter of either construct is inducible)
(d) causing or permitting transcription of the expression cassettes such as to cause silencing of the target gene in an organism.
   Plants and Methods of Transformation

In preferred embodiments the invention is performed (i.e. the DNA construct is used) in order to silence a target endogenous gene in a plant which is transformed with said construct. The invention is applicable to both monocot and dicot plants.

Thus in one aspect there is provided a method of silencing a target gene in an organism, which method comprises the steps of:
(a) providing the organism transformed with a DNA construct including an expression cassette comprising (i) a promoter, operably linked to (ii) a chimeric nucleotide sequence encoding all are part of the target gene and a transgene, and where appropriate causing or permitting transcription of the chimeric sequence,
(b) initiating PTGS of said transgene in the organism using any of the methods described herein such as to cause silencing of the target gene in the organism.

The present invention may be used in plants such as crop plants, including cereals and pulses, maize, wheat, potatoes, tapioca, rice, sorgum, millet, cassaya, barley, pea and other root, tuber or seed crops. Important seed crops are oil seed rape, sugar beet, maize, sunflower, soybean and sorghum. Horticultural plants to which the present invention may be applied may include lettuce, endive and vegetable brassicas including cabbage, broccoli and cauliflower, and carnations and geraniums. The present invention may be applied to tobacco, cucurbits, carrot, strawberry, sunflower, tomato, pepper, chrysanthemum, poplar, eucalyptus and pine.

For genomic analysis (see below), it may be preferred to use Nicotiana benthamiana, Arabidopsis thaliana or Oryza sativa.

Any appropriate method of plant transformation may be used to generate plant cells containing a construct within the genome in accordance with the present invention. Following transformation, plants may be regenerated from transformed plant cells and tissue. Successfully transformed cells and/or plants, i.e. with the construct incorporated into their genome, may be selected following introduction of the nucleic acid into plant cells, optionally followed by regeneration into a plant, e.g. using one or more marker genes such as antibiotic resistance.

All the following methods may also be used analogously to further (or earlier) transform plants with the transgene-silencing constructs (e.g. amplicon) in embodiments in which they are used.

Plants transformed with the DNA construct may be produced by standard techniques which are already known for the genetic manipulation of plants. DNA can be transformed into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser—see attached) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d). Physical methods for the transformation of plant cells are reviewed in Oard, 1991, Biotech. Adv. 9: 1-11.

Agrobacterium transformation is widely used by those skilled in the art to transform dicotyledonous species. Production of stable, fertile monocot transgenic plants may be achieved e.g. using the techniques of, or analogous to, Toriyama, et al. (1988) Bio/Technology 6, 1072-1074; Zhang, et al. (1988) Plant Cell Rep. 7, 379-384; Zhang, et al. (1988) Theor Appl Genet 76, 835-840; Shimamoto, et al. (1989) Nature 338, 274-276; Datta, et al. (1990) Bio/Technology 8, 736-740; Christou, et al. (1991) Bio/Technology 9, 957-962; Peng, et al. (1991) International Rice Research Institute, Manila, Philippines 563-574; Cao, et al. (1992) Plant Cell Rep. 11, 585-591; Li, et al. (1993) Plant Cell Rep. 12, 250-255; Rathore, et al. (1993) Plant Molecular Biology 21, 871-884; Fromm, et al. (1990) Bio/Technology 8, 833-839; Gordon-Kamm, et al. Plant Cell 2, 603-618; D'Halluin, et al. (1992) Plant Cell 4, 1495-1505; Walters, et al. (1992) Plant Molecular Biology 18, 189-200; Koziel, et al. (1993) Biotechnology 11, 194-200; Vasil, I. K. (1994) Plant Molecular Biology 25, 925-937; Weeks, et al. (1993) Plant Physiology 102, 1077-1084; Somers, et al. (1992) Bio/Technology 10, 1589-1594; WO92/14828). In particular, Agrobacterium mediated transformation is now emerging also as an highly efficient transformation method in monocots (Hiei et al. (1994) The Plant Journal 6, 271-282).

The generation of fertile transgenic plants has been achieved in the cereals rice, maize, wheat, oat, and barley (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

Microprojectile bombardment, electroporation and direct DNA uptake are preferred where Agrobacterium is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

Following transformation, a plant may be regenerated, e.g. from single cells, callus tissue or leaf discs, as is standard in the art. Almost any plant can be entirely regenerated from cells, tissues and organs of the plant. Available techniques are reviewd in Vasil et al., Cell Culture and Somatic Cel Genetics of Plants, Vol I, II and III, Laboratory Procedures and Their Applications, Academic Press, 1984, and Weissbach and Weissbach, Methods for Plant Molecular Biology, Academic Press, 1989.

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

In a further aspect of the present invention there is disclosed a host cell including, or transformed by, the DNA construct according to the present invention. Use of the construct as described above in the transformation (stable or transient) of a plant is also embraced by the invention. The host cell will preferably have incorporated into its genome a construct as described above (i.e. be transformed by it). Also according to the invention there is provided a plant cell having incorporated into its genome a DNA construct as disclosed. A further aspect of the present invention provides a method of making such a plant cell involving introduction of a vector including the construct into a plant cell. Such introduction should be followed by recombination between the vector and the plant cell genome to introduce the sequence of nucleotides into the genome. RNA encoded by the introduced nucleic acid construct may then be transcribed in the cell and descendants thereof, including cells in plants regenerated from transformed material. A gene stably incorporated into the genome of a plant is passed from generation to generation to descendants of the plant, so such descendants should show the desired phenotype.

The present invention also provides a plant comprising a plant cell as disclosed. In addition to a plant, the present invention provides any clone of such a plant, seed, selfed or hybrid progeny and descendants, and any part of any of these, such as cuttings, seed.

Methods of Identifying Gene Function

A further aspect of the present invention provides a method of reducing or suppressing or lowering the level of a target gene in a plant cell, the method including causing or allowing transcription from the construct as disclosed above.

In preferred forms the present invention is concerned with providing silencing-based methods are useful in functional genomics. Thus in one aspect of the present invention, the target gene may be of unknown phenotype, in which case the system may be employed to analyse the phenotype by generating a widespread null (or nearly null) phenotype. The target gene may be essential, which is to say that the null phenotype is lethal to the cell or tissue in question.

In preferred embodiments, plants are first transformed with a single type of “triggering construct” (e.g. based on an inducible promoter and Amplicon or IR as described above). After that point only simple high throughput cloning steps are needed. A cDNA (or other) library can be cloned into a corresponding “spreading construct”. This library is then used for large scale plant transformations in order to generate an extensive collection of transformant plants. Following induction of the triggering construct, and due to the dominant character of RNA silencing, these plants can be immediately screened for specific null-phenotypes. The silenced gene sequences of interest in the phenotypes can be PCR-amplified easily by using primers specific to the spreading construct (and in particular the portion therein in which the library insert is introduced). This system provides an excellent inducible and dominant null-phenotype generating machine for high throughput forward genetic studies in A. thaliana.

Furthermore, due to the dsRNA synthesis during the spreading phenomenon, the antisense siRNAs needed to target endogenous mRNA will be produced independently of the orientation of the endogenous cDNA in the spreading construct. This further facilitates the cloning steps.

This aspect of the invention may comprise a method of characterizing a target gene comprising the steps of:
(a) silencing the target gene in a part or at a certain development stage of the plant using the system described above,
(b) observing the phenotype of the part of the plant in which, or when, the target gene has been silenced.

Thus in one embodiment the invention provides a method of characterizing a target gene comprising the steps of:
(a) providing a first DNA construct (“spreading constuct”) including an expression cassette comprising (i) a promoter, operably linked to (ii) a chimeric nucleotide sequence encoding all are part of the target gene and a transgene,
(b) providing a plant transformed with a second DNA construct (“triggering construct”) capable of triggering silencing of the transgene,
(c) transforming the organism with said second DNA construct such that the expression cassette is inserted into the genome,
(d) causing or permitting transcription of the expression cassettes such as to cause silencing of the target gene in an organism.

(e) observing the phenotype of the plant in which the target gene has been silenced.

In preferred embodiments the silencing is initiated using an inducible promoter e.g. applying an exogenous inducer to cause transcription of the triggering construct at an appropriate time. The phenotype is then observed.

Generally the observation will be contrasted with a plant wherein the target gene is being expressed in order to characterise (i.e. establish one or more phenotypic characteristics of) the gene.

In a further aspect there is disclosed a method of altering the phenotype of a plant comprising use of the silencing method discussed above. Traits for which it may be desirable to change the phenotype include the following: colour; disease or pest resistance; ripening potential; male sterility etc.

Detecting Target Silencing

In addition to monitoring phenotype, the methods of the present invention may be followed by assessing the silencing of the target gene.

Detection may be using any method known in the art or described herein. Detection may be by analysis of siRNAs and may involve the steps of:
(i) obtaining sample material from the organism,
(ii) extracting nucleic acid material therefrom,
(iii) analysing the extracted nucleic acid in order to detect the presence or absence of siRNAs therein corresponding to the target gene. The result of the analysis in step (iii) may be correlated with the presence of silencing in the organism.

The ‘sample’ may be all or part of the organism, but will include at least some cellular material.

Alternatively it may be preferred to investigate methylation of the target sequence.

The invention will now be further described with reference to the following non-limiting Figures and Examples. Other embodiments of the invention will occur to those skilled in the art in the light of these.

### FIGURES, SEQUENCE APPENDIX AND EXAMPLES

### FIGURES

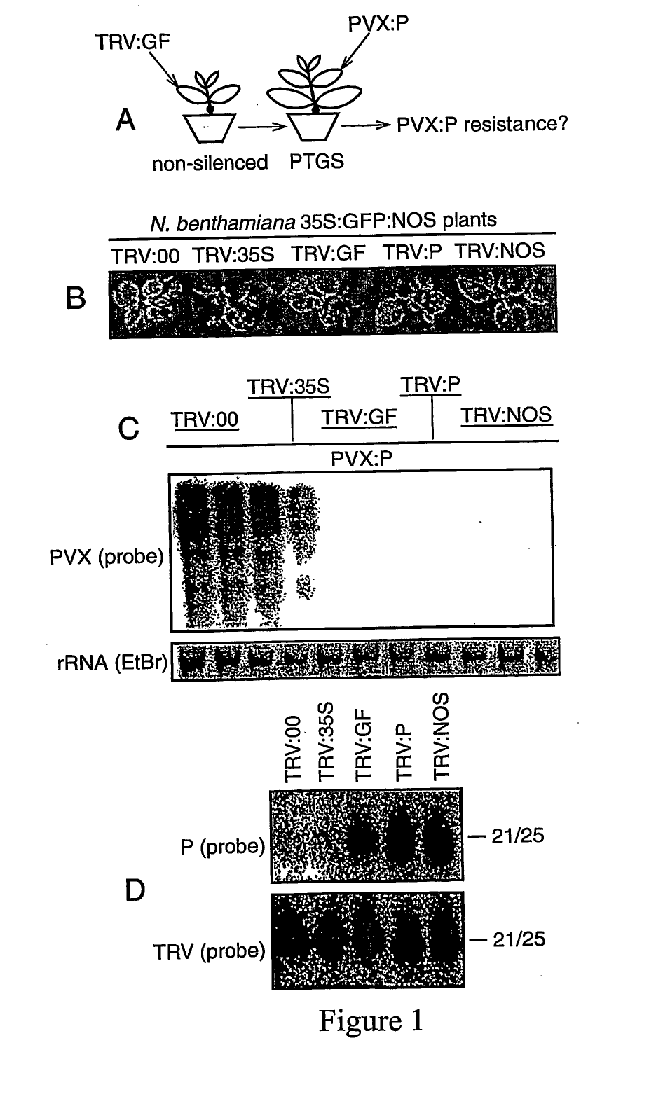

FIG. 1. Spreading of targeting on a GFP transgene.

(A) Schematic representation of the experimental procedure. Post-transcriptional gene silencing (PTGS) of a 35S:GFP:NOS transgene in N. benthamiana plants (line 16c) was triggered by inoculating with TRV carrying a region of the GFP sequence. Subsequently plants were challenge-inoculated with PVX carrying another region of the GFP transgene and resistance was assessed. Plants infected with TRV:00 or TRV:35S were used as non-PTGS controls. (B) TRV:00-, TRV:35S-, TRV:GF-, TRV:P- and TRV:NOS-infected 16c plants photographed at 21 dpi under ultra violet light. Silencing of GFP is evident as red fluorescence which is due to the chlorophyll. Infection with TRV:35S induces transcriptional gene silencing (TGS) of the transgene. (C) PVX RNA levels in TRV-infected plants challenge-inoculated with PVX:P. RNA samples were extracted from upper leaves of PVX:P-challenged plants at 10 dpi and a probe specific for PVX was used in Northern blot analysis. At this time point TRV:00-infected plants were not yet showing GFP silencing by PVX:P. Ethidium bromide (EtBr) stained rRNAs are shown in the lower panel. (D) Analysis of siRNAs (21/25 nucleotide in length) from TRV-inoculated plants at 21 dpi. Sense RNA probes were specific for antisense RNAs corresponding to the P region of GFP (top panel) or TRV (bottom panel).

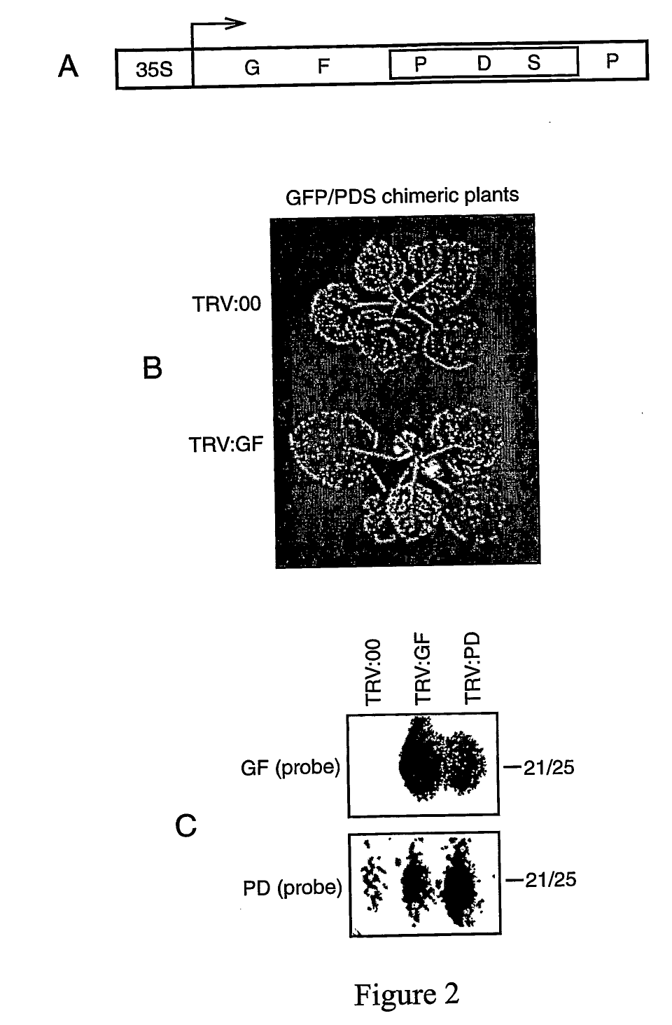

FIG. 2. Spreading of targeting on a GFP/PDS chimeric gene.

(A) Schematic representation of the GFP/PDS chimeric transgene including the 35S promoter, the GFP sequences and the PDS region. (B) GFP/PDS chimeric plants (line #D) inoculated with TRV:00, or TRV:GF photographed at 21 dpi. (C) Analysis of siRNAs (21/25 nucleotide in length) from TRV:GF- or TRV:PD-infected at 21 dpi. Sense RNA probes were specific for antisense RNAs corresponding to the GF region of GFP (top panel) or PD of the PDS (bottom panel).

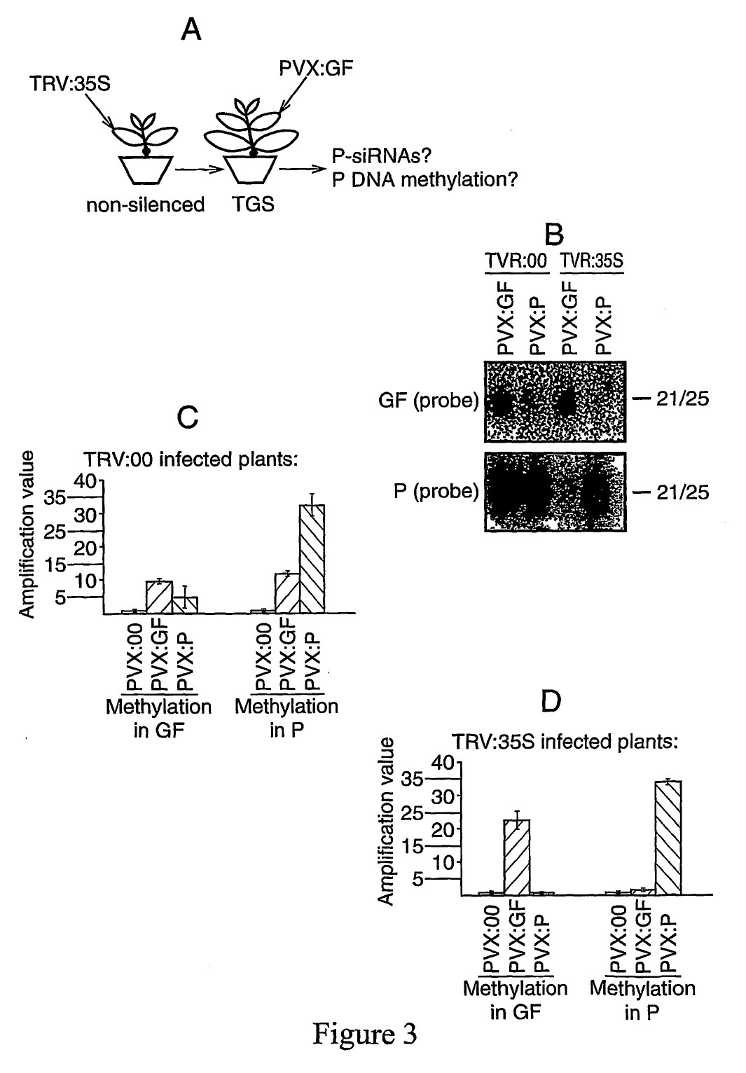

FIG. 3. Spreading requires transgene transcription.

(A) Schematic representation of the experimental procedure. Transcriptional gene silencing (TGS) of a 35S:GFP:NOS transgene in N. benthamiana (line 16c) was triggered by TRV:35S-infection. Following the onset of TGS, plants were inoculated with PVX carrying a region of the GFP transgene and siRNAs and DNA methylation corresponding to the rest of the GFP sequence were assessed. Plants infected with TRV:00 were used as a non-TGS control. (B) Analysis of siRNAs (21/25 nucleotide in length) from non-TGS (TRV:00) or TGS (TRV:35S) plants infected with PVX:GF or PVX:P. RNA samples were extracted from upper leaves at 21 days post-PVX infection. At this time point TRV:00-infected control plants were showing full GFP silencing by PVX:GF or PVX:P. Sense RNA probes were specific for antisense RNAs corresponding to the GF or P regions of GFP (top and bottom panel respectively). (C and D) Analysis of DNA methylation within GF and P regions by Sau96I digestion followed by quantitative PCR. DNA samples were prepared from non-TGS (TRV:00) (C) or TGS (TRV:35S) (D) plants infected with PVX:00, PVX:GF or PVX:P at 21 dpi. Amplification values represent the degree of DNA methylation. Values are the average of three independent experiments. The values represent the DNA methylation level: the higher the amplification value, the greater the degree of DNA methylation in the PCR template.

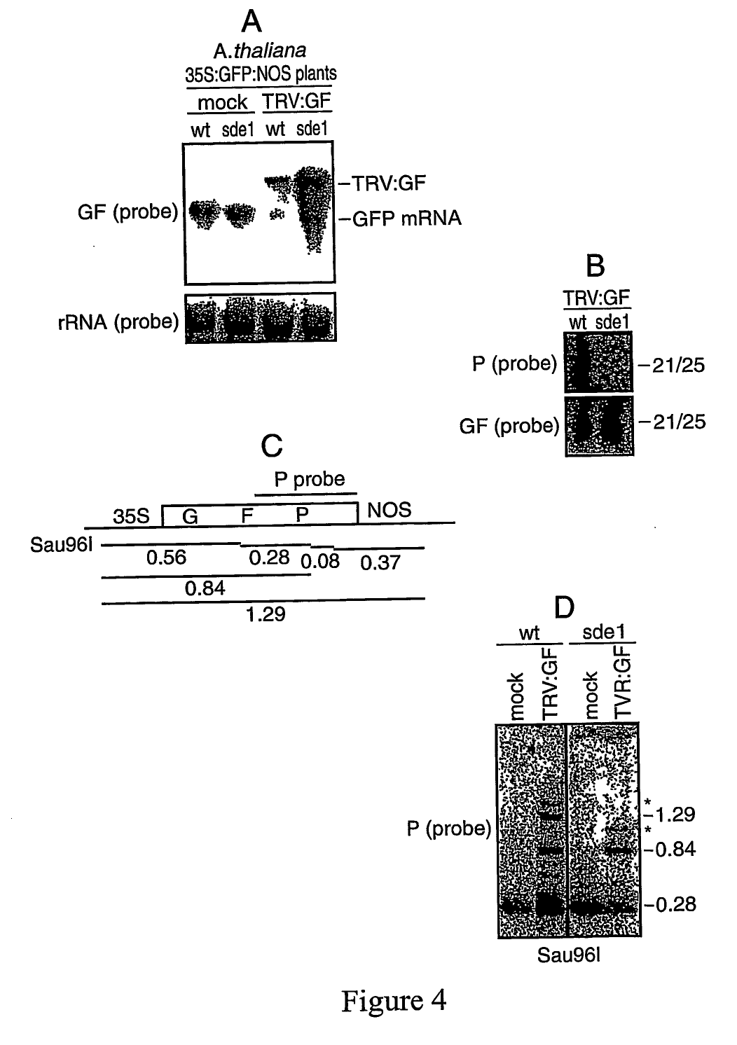

FIG. 4. Spreading requires SDE1SGS2.

(A) Detection of GFP and TRV:GF RNAs in mock- or TRV:GF-inoculated wild-type (wt) or sde1/sgs2 mutant (sde1) 35S:GFP:NOS A. thaliana plants (top panel). RNA preparations were made from pools of 10 plants at 7-10 dpi and the probe used was specific to the GF region of the GFP RNA. Ethidium bromide (EtBr) stained rRNAs are shown in the lower panel. (B) Analysis of siRNAs (21/25 nucleotide in length) in wild-type (wt) and sde1/sgs2 TRV:GF-inoculated plants at 7-10 dpi. Sense RNA probes were specific for antisense RNAs corresponding to the GF or P regions of GFP (low and top panel respectively). (C) Schematic map of the 35S:GFP:NOS transgene in A. thaliana. Expected sizes (in kb) for total and relevant partial Sau96I restriction enzyme digestions and the P probe used for Southern analysis are indicated. (D) Southern blot analysis of Sau96I digested DNA extracted from pooled plants as described in (A). Sizes of relevant DNA fragments are indicated. Bands marked with an asterisk are due to a low level of methylation at the Sau96I site within the 35S promoter.

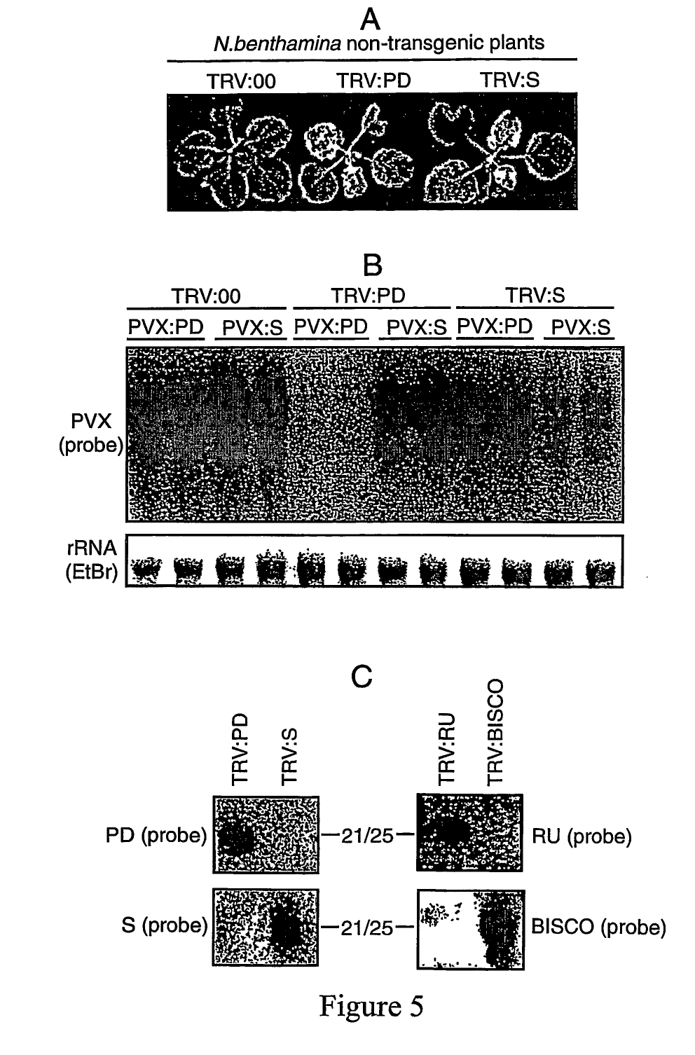

FIG. 5. Spreading and endogenes.

(A) N. benthamiana non-transgenic plants inoculated with TRV:00, TRV:PD or TRV:S photographed at 21 dpi. (B) PVX RNA levels in TRV:00-, TRV:PD- or TRV:S-infected plants challenge-inoculated with PVX:PD or PVX:S. RNA samples were extracted from PVX-inoculated leaves at 4 dpi and a probe specific for PVX was used in Northern blot analysis. Ethidium bromide (EtBr) stained rRNAs are shown in the lower panel. (C) Analysis of siRNAs (21/25 nucleotide in length) from TRV:PD-, TRV:S-, TRV:RU- or TRV:BISCO-inoculated plants at 21 dpi. Sense RNA probes were specific for antisense RNAs corresponding to the PD, S, RU and BISCO regions of the PDS and rubisco genes.

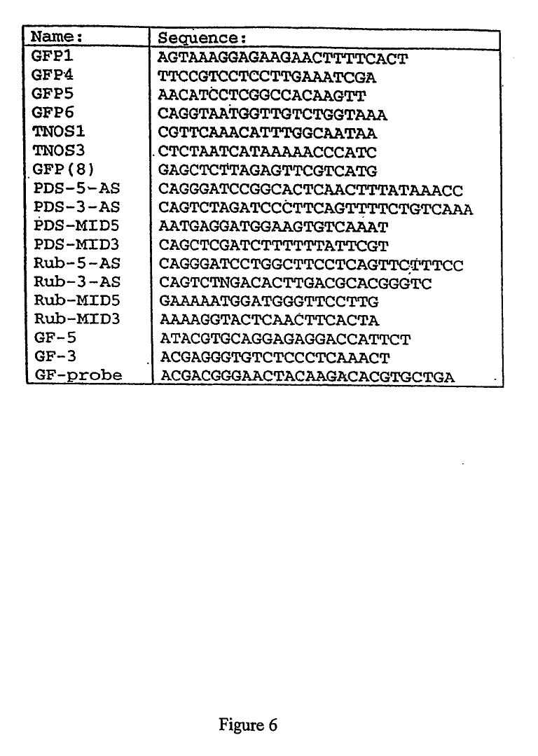

FIG. 6. This shows Table 1 listing the sequences of the oligonucleotides used in the Examples.

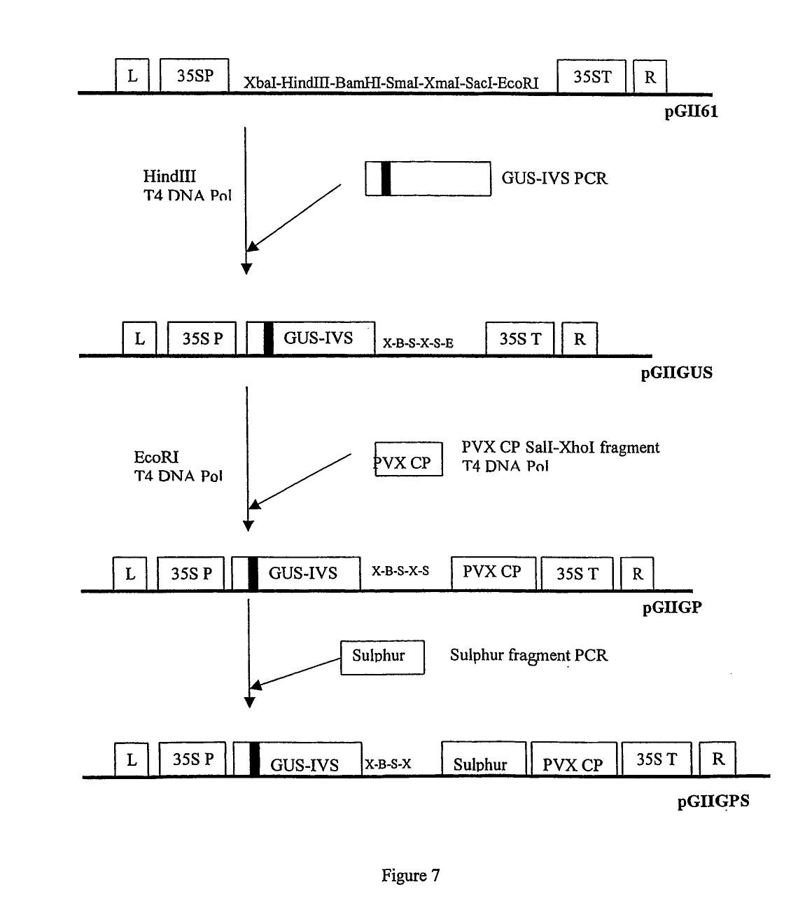

FIG. 7. Construction of pGIIGPS. L: left border of T DNA. R: right border. 35S P: 35S promoter. 35S T: 35 S terminator. IVS: intron from ST-LS1 gene from potato cloned in GUS. Showed as a black box.

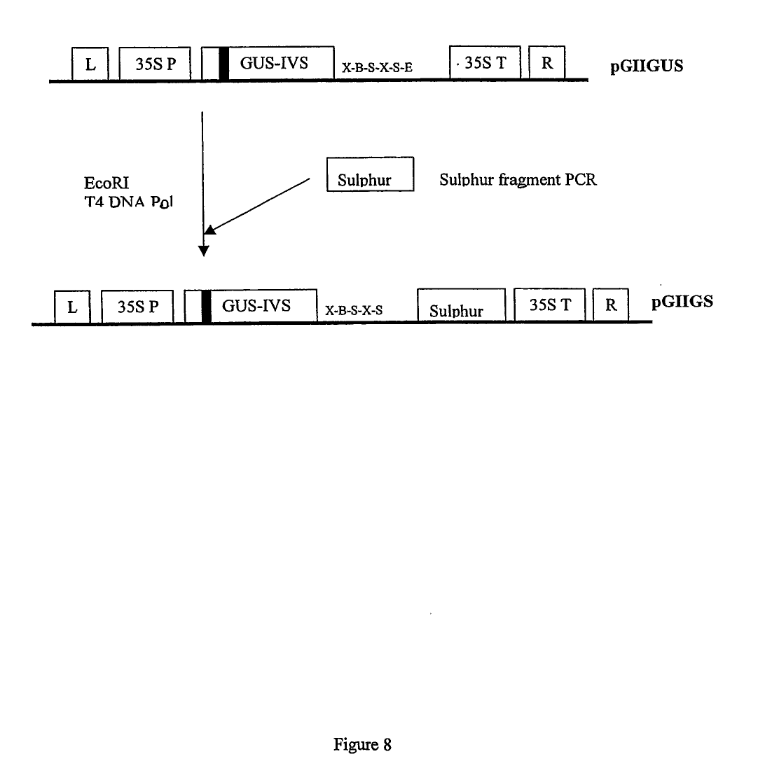

FIG. 8. Construction of negative control pGIIGS. Abbreviations as in FIG. 7.

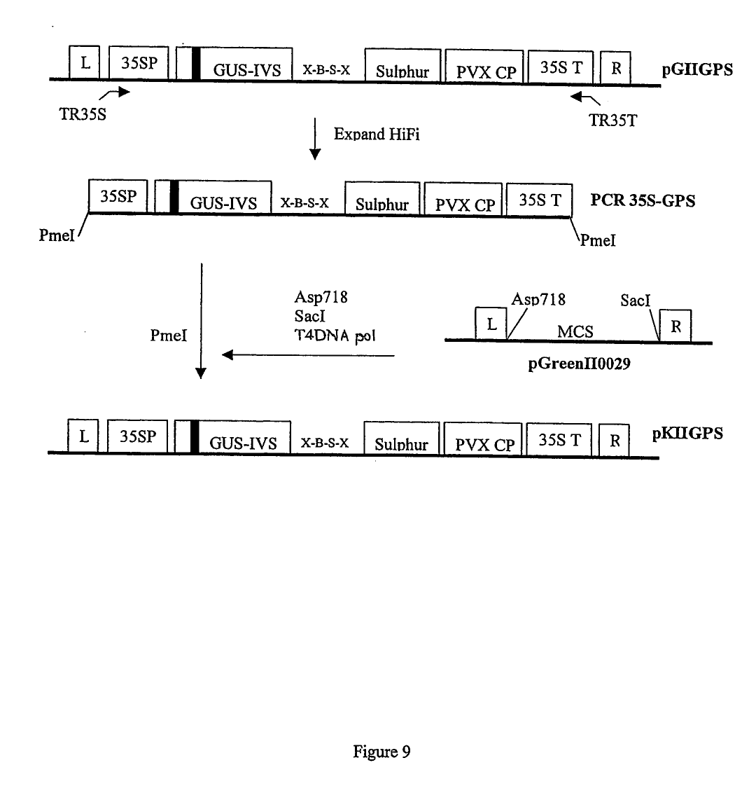

FIG. 9. Construction of pKIIGPS. Abbreviations as in FIG. 7.

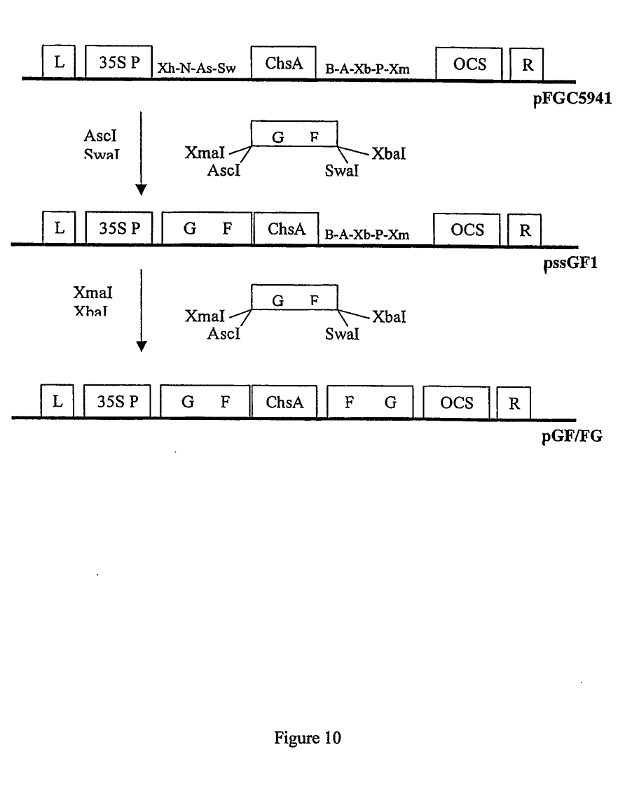

FIG. 10. Construction of pGF/FG. ChsA: intron from Chalcone Sintase gene. OCS: octopine synthase 3′ polyadenylation sequence. Xh: XhoI. N: NcoI. As: AscI. Sw: SwaI. B: BamHI. A: AvrII. Xb: XbaI. P: PacI. Xm: XmaI.

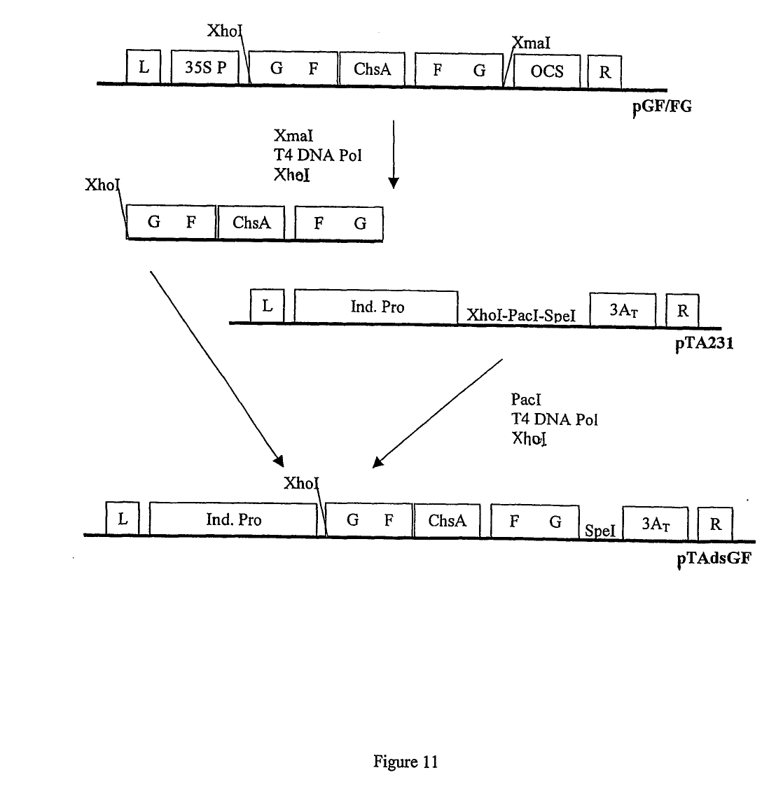

FIG. 11. Construction of pTAdsGF. Ind Pro: stands for the elements of the inducible promoter. 3A_{T}: Pea Rubisco small subunit gene terminator.

### SEQUENCE APPENDIX

1 Sequence of pKIIGPS.
2 Sequence of pKIIGS. 35S cassette with GUS and sulphur inserts.
3 Sequence of pGF/FG. Showed from promoter to terminator.
4 Inducible promoter and hairpin GF construct in pTAdsGF

### EXAMPLES

Experimental Procedures

Use of genetically modified plant viruses was licensed by MAFF license PHL 24B/3654 (3/2001).

Transgenic Plants

The N. benthamiana 16c line and the A. thaliana GFP wild-type and sde1/sgs2 lines (wt[G] and sde1(GI) were described previously (Dalmay et al., 2000a; Dalmay et al., 2001; Ruiz et al., 1998). Visual observation of GFP fluorescence was performed according to Voinnet et al. (1998). The line #D was made as follows: a 600 bp GFP DNA fragment was PCR amplified using oligos GFP1 and GFP6 (see Table 1 for sequences) and cloned into the SmaI-digested pJIT60 vector (www.pgreen.ac.uk). Then, the KpnI/XhoI fragment was inserted into KpnI/XbaI-digested pGreen0229 binary vector (www.pgreen.ac.uk). A 416 bp N. benthamiana PDS cDNA fragment was PCR amplified using oligos PDS-5-AS and PDS-3-AS (see Table 1 for sequences) and cloned in the antisense orientation into the Bst1107I site of GFP. The resulting vector was used to obtain transgenic N. benthamiana according to Horsch et al. (Horsch et al., 1985).

Viral Vectors and Virus Inoculations

The primers used to PCR amplify the different regions of the GFP:NOS, PDS and rubisco genes (and the sizes of the PCR products) are as follows: GFP1 and GFP4 for GF (400 bp), GFP5 and GFP8 for P (332 bp), TNOS1 and TNOS3 for NOS (154 bp), PDS-5-AS and PDS-MID3 for PD (213 bp), PDS-MID5 and PDS-3-AS for S (216 bp), Rub-5-AS and Rud-MID3 for RU (272 bp) and Rub-MID5 and Rub-3-AS for BISCO (250 bp) (see Table 1 for sequences). These fragments were cloned into pGEM-Teasy (Promega), excised with SalI/ApaI and inserted into SalI/ApaI-digested pTV.00 (Ratcliff et al., 2001) to produce pTRV:GF, pTRV:P, pTRV:NOS, PTRV:PD, pTRV:S, pTRV:RU and PTRV:BISCO, respectively. The TRV:35S vector was described previously (Jones et al., 2001). TRV inoculations of N. benthamiana have been described previously (Ratcliff et al., 2001). For A. thaliana TRV:GF inoculations, 7 day old seedlings were vacuum-infiltrated with a pTRV:GF/pBINTRA6 Agrobacterium suspension mix (Ratcliff et al., 2001). pPVX:PD and pPVX:S vectors were obtained by cloning the PD and S fragments into the SmaI site of pGR107 (Jones at al., 1999). PVX:GF and PVX:P vectors were described previously (Ruiz et al., 1998; Voinnet et al., 1998). PVX inoculations of N. benthamiana were as described in Ruiz et al. (1998), Voinnet et al. (1998) and Jones et al. (1999).

Nucleic Acid Analysis

RNA was extracted using Tri-reagent (Sigma) according to the manufacturer's instructions. Total RNA was used for both high molecular weight RNA and siRNA analysis. Northern blot analysis were performed as described previously (Jones et al., 1998); siRNA analyses were performed as described in Hamilton & Baulcombe (1999). For siRNAs detection, probes were made by in vitro transcription from pKS (Stratagene) carrying the corresponding fragments cloned into the SmaI site. The TRV probe corresponds to a fragment of the TRV coat protein obtained by EcoRI digestion of pTV.00 (Ratcliff et al., 2001) and cloned into pKS. Genomic DNA was extracted using the DNeasy plant DNA extraction kit (Qiagen) according to the manufacturer's instructions. DNA gel-blot was performed as described previously (Jones et al., 1998). The DNA methylation analysis by Sau96I digestion and Taqman quantitative PCR was performed as described previously (Jones et al., 2001) using DNA prepared from upper leaves. The two oligos and the probe used for the analysis of the GF region were GF-5, GF-3, and GF-probe respectively (see Table 1 for sequences). The oligos and probes used for analysis of the P region and controls were described previously (Jones et al., 2001).

### Example 1

### Spreading of Targeting on a GFP Transgene Induced by VIGS

The distribution of RNA silencing targets in VIGS had not previously been investigated.

Therefore we carried out VIGS of a 35S:GFP:NOS transgene in N. benthamiana (line 16c) (Ruiz et al., 1998) using tobacco rattle virus (TRV) vectors carrying inserts corresponding to different parts of the transgene. We then monitored the RNA target of VIGS by challenge-inoculating plants with potato virus X (PVX) vectors carrying other parts of the GFP transgene (FIG. 1A) and by characterizing the siRNAs associated with silencing of the GFP mRNA. From our previous experiments we anticipated that the PVX RNA would not accumulate if it carries an insert that is target of RNA silencing. Accordingly, there would be accumulation of siRNAs corresponding to the target region of RNA silencing.

The initiator constructs in these experiments were TRV vectors carrying the 5′ or 3′ halves of the GFP coding region or the 3′ untranslated region (TRV:GF, TRV:P and TRV:NOS, respectively). As controls, plants were infected with TRV without an insert (TRV:00) or carrying the 35S promoter (TRV:35S). TRV:00 would not cause silencing of the transgene whereas TRV:35S would trigger transcriptional silencing of 35S-driven transgenes (Jones et al., 1999; Jones et al., 2001).

By 21 days post inoculation (dpi) there was loss of green fluorescence, indicative of RNA silencing, in plants infected with TRV:35S, TRV:GF, TRV:P and TRV:NOS whereas plants infected with TRV:00 remained fully green fluorescent (FIG. 1B). Correspondingly, there was less GFP mRNA in silenced plants than in non-silenced plants (data not shown). These TRV-infected plants (21 dpi) were then challenge-inoculated with PVX:P (a PVX vector modified to carry the same 3′ part of the GFP sequence that is present in TRV:P) and levels of PVX viral RNA were assessed 10 days later. FIG. 1C shows that PVX:P accumulated at high levels in TRV:00 and TRV:35S infected plants and at low levels in TRV:GF, TRV:P or TRV:NOS infected plants. Thus the ‘P’ region of GFP was a target irrespective of whether the initiator was GF, P or NOS.

To characterize siRNAs we used a sense probe specific for the antisense 3′ part of GFP (P probe) and we sampled the TRV-infected tissues at 21 dpi. As shown in FIG. 1D (top panel), antisense P-specific siRNAs (P-siRNAs) were present in samples from TRV:GF-, TRV:P- and TRV:NOS-infected plants but not in TRV:00- and in TRV:35S-infected plants. Similarly, GF-siRNAs and NOS-siRNAs were present in samples from TRV:GF-, TRV:P- and TRV:NOS-infected plants but not in those from TRV:00- and in TRV:35S-infected plants (data not shown). Thus, irrespective of whether the initiator sequence was GF, P or NOS, the siRNA population was distributed throughout the transcribed region of the GFP transgene. TRV-siRNAs were detected in all the TRV-infected plants (FIG. 1D, bottom panel) as expected from the finding that RNA silencing is a natural mechanism for virus resistance in plants (Hamilton and Baulcombe, 1999; Ratcliff et al., 1999).

These combined results demonstrate that the target of RNA silencing can spread within the transcribed regions of the transgene from the initiator region in both 3′ (from GF to P) and 5′ (from NOS to P) directions. Moreover, because NOS-siRNAs were produced following initiation with GF and vice versa, we have shown that spreading can extend further than the 332 bp corresponding to the P region. The presence of antisense siRNAs corresponding to sequences beyond the initiator region indicates that a dsRNA copy of the target has been produced. The absence of siRNAs corresponding to the coding region of the transgene in TRV:35S-infected plants correlates with our previous finding that 35S DNA methylation in TRV:35S-infected plants does not spread into the transcribed regions (Jones et al., 1999).

### Example 2

### Spreading of Targeting on a GFP/PDS Chimeric Transgene Using VIGS

We also investigated spreading of targeting in N. benthamiana carrying a chimeric GFP/PDS gene (line #D). This chimeric gene is composed of the 35S promoter driving transcription of a GFP sequence with an insertion of 429 bp of the N. benthamiana phytoene desaturase (PDS) cDNA (FIG. 2A). Inhibition of PDS causes suppression of carotenoid biosynthesis and susceptibility to photobleaching (Demmig-Adams and Adams, 1992; Kumagai et al., 1995; Ruiz et al., 1998).

It was believed that siRNAs corresponding to the PDS region of the chimeric gene (PDS-siRNAs) may be produced by spreading of targeting from a GFP initiator. These PDS-siRNAs would target the endogenous PDS mRNA and cause photobleaching.

To test these predictions line #D plants were inoculated with TRV:00 or TRV:GF and monitored for photobleaching. FIG. 2B shows that TRV:GF but not TRV:00 triggered photobleaching. Non-transgenic or 35S:GFP:NOS transgenic plants did not show any PDS silencing after infection with TRV:GF (data not shown). Spreading of targeting in line #D was confirmed by analyzing the siRNA population following infection with TRV:GF or TRV:PD which carries a part of the PDS region of the chimeric transgene. Both GF- and PD-siRNAs were present in TRV:GF- or TRV:PD-infected plants but were not in TRV:00-infected plants (FIG. 2C). Therefore spreading of targeting in both 5′ and 3′ directions is not specific to the 35S:GFP:NOS transgene in line 16c.

### Example 3

### Spreading and Transgene Transcription Using VIGS

To determine whether spreading of targeting depends on transgene transcription we carried out experiments after transcriptional silencing of the 35S:GFP:NOS transgene. This transcriptional silencing was induced by infection with TRV:35S, as described previously (Jones et al., 1999; Jones et al., 2001). After 21 days these silenced plants were inoculated with PVX:GF, PVX:P or PVX:00 (a PVX vector without GFP inserts). Spreading of targeting and DNA methylation was assessed 21-28 days later (FIG. 3A). As a control, the same PVX vectors were inoculated to TRV:00-infected plants.

As shown in FIG. 3B (top and bottom panels) both GF- and P-siRNAs were produced in TRV:00-infected plants following PVX:GF or PVX:P inoculations. Thus, the spreading of targeting occurred with a PVX vector as for TRV vectors, and was not affected by the presence of TRV:00. In contrast, in TRV:35S-infected plants, only P-siRNAs were detected in PVX:P-infected tissue and, likewise, only GF-siRNAs were detected in PVX:GF-infected tissue (FIG. 3B, top and bottom panels). Thus, from the lack of spreading in TRV:35S-infected plants we conclude that spreading requires transcription of the target.

Spreading of GFP DNA methylation was assessed by Sau96I digestion followed by quantitative PCR (TaqMan, Applied Biosystems) of the GF and P regions of the GFP transgene. Sau96I is a methylation-sensitive restriction enzyme that cleaves within the GF and P sequences. Methylation at these Sau96I sites would prevent digestion and result in a higher level of amplifiable DNA than in non-methylated samples. Thus, the higher the amplification value, the greater the degree of DNA methylation in the PCR template.

FIG. 3C shows that, after PVX:GF or PVX:P inoculation of TRV:00-infected plants, amplification values for both GF and P sequences were higher than those of the non-methylated negative control (PVX:00-infected plants). Thus, when the 35S:GFP:NOS transgene is transcribed, DNA methylation is detected not only in the region targeted but also in adjacent sequences. However, the level of DNA methylation in GF was higher after PVX:GF infection than after PVX:P infection, and vice versa. In contrast, when the transgene was transcriptionally silenced by TRV:35S, methylation was only detected in the GFP region being targeted by the recombinant PVX vector (FIG. 3D). Thus, methylation was restricted to GF after PVX:GF infection, and to P after PVX:P infection. Therefore spreading of DNA methylation, as for targeting, is dependent on transcription of the 35S:GFP:NOS transgene.

### Example 4

### Spreading and SDE1/SGS2 with VIGS

In order to determine whether SDE1/SGS2 is required for the spreading of targeting and DNA methylation we carried out experiments in wild-type or sde1/sgs2 A. thaliana carrying a 35S:GFP:NOS transgene. RNA silencing was initiated with TRV:GF and nucleic acid samples were taken at 10-15 dpi when GFP silencing was visible in both wild-type and mutant plants (data not shown). In wild-type plants, the GFP silencing was maintained throughout the life of the plant. However, as reported previously, the GFP silencing was only transient in the sde1/sgs2 background and the older plants had fully green fluorescent leaves (Dalmay et al., 2001).

FIG. 4A shows that, at 10-15 dpi, GFP mRNA levels were lower in silenced plants than in non-silenced mock inoculated plants and that TRV:GF RNA was more abundant in sde1/sgs2 than in the wild-type plants. The GF-siRNAs were present in both samples but were more abundant in sde1/sgs2 plants (FIG. 4B, bottom panel). This increased abundance of GF-siRNA is most likely due to the higher accumulation of TRV:GF in sde1/sgs2 plants (FIG. 4A). From these results we conclude that SDE1/SGS2 is not necessary for siRNA production from the TRV:GF RNA. The P-siRNAs were detected in the TRV:GF-infected wild-type plants (FIG. 4B, top-panel) indicating that spreading of targeting takes place in A. thaliana as in N. benthamiana. However, P-siRNAs were not produced in the TRV:GF-infected sde1/sgs2 plants (FIG. 4B top panel). Therefore spreading of targeting is dependent on SDE1/SGS2.

GFP DNA methylation in TRV:GF-infected A. thaliana was assessed by Southern blot analysis after Sau96I digestion and hybridization with a P-specific probe. FIG. 4C shows the organization of the 35S:GFP:NOS transgene, the location of Sau96I restriction enzyme sites and the sizes of total and relevant partial digestion products of the GFP transgene. FIG. 4D shows that, in mock-inoculated plants, only the 0.28 kb fragment (corresponding to the unmethylated DNA) could be detected. The 0.37 kb and 0.08 kb fragments of were most likely not detected because of either the low resolution of the gel or because the P probe overlapping region is too short.

In TRV:GF-infected wild-type plants the Sau96I digestion products were 0.28 kb, 0.84 kb and 1.29 kb. The 0.84 kb fragment indicates methylation in the GF region and the 1.29 kb fragment reflects methylation in both GF and P regions. In the TRV:GF-infected sde1/sgs2 plants the only fragment diagnostic of transgene methylation was 0.84 kb. From these results we conclude that spreading of DNA methylation, like spreading of targeting, is dependent on SDE1/SGS2. Results leading to the same conclusion were also generated with HaeIII digested DNA (data not shown).

### Example 5

### Spreading and Endogenous Genes

VIGS of PDS and ribulose bisphosphate carboxylase small subunit (rubisco) is unlike that of GFP. This RNA silencing of these endogenous genes is dependent on the continuous presence of the virus and the target DNA is not methylated (Jones et al., 1999; Ruiz et al., 1998; Thomas et al., 2001). Thus, VIGS of PDS and rubisco does not show the transition to the maintenance phase of RNA silencing. To assess spreading of targeting in VIGS of PDS we used the same approach as with the 35S:GFP:NOS transgene (FIG. 1A). First we initiated VIGS in N. benthamiana with TRV vectors carrying a fragment of the PDS gene. We then challenge-inoculated with PVX carrying a different part of the PDS gene. Spreading of targeting would have caused the plants to be resistant against the challenge inoculum.

The TRV vectors used in these experiments were TRV:PD and TRV:S which carry two contiguous non-overlapping regions of the N. benthamiana PDS cDNA. Plants infected with TRV:00 were used as a control for non-specific effects of virus inoculation. By 21 dpi, PDS silencing was observed as photobleached tissues in plants infected with both TRV:PD and TRV:S. In contrast, plants infected with TRV:00 remained non-silenced (FIG. 6A). The TRV-infected plants were then challenge-inoculated with PVX:PD or PVX:S (carrying the PDS fragments of TRV:PD and TRV:S respectively), and levels of viral PVX RNA were assessed four days later by northern blotting. FIG. 6B shows that, in TRV:00-infected plants, both PVX:PD and PVX:S accumulated at high levels. In TRV:PD-infected plants PVX:S accumulated at high levels. In contrast, PVX:PD accumulated at low levels as a consequence of crossprotection (Ratcliff et al., 1997; Ratcliff et al., 1999). Conversely, in TRV:S-infected plants PVX:PD accumulated at high levels and PVX:S at low levels. From these data we conclude that spreading of targeting had not occurred. Similar results were obtained when the endogenous target gene was the highly expressed rubisco gene (data not shown).

To further investigate spreading of targeting we characterized the antisense siRNA population in plants infected with the TRV vectors. As shown in FIG. 6C (left panels), at 21 dpi, PD-siRNAs were present in samples from TRV:PD-infected plants and absent in TRV:S-silenced plants. Likewise, S-siRNAs were detected in TRV:S-infected plants but not in TRV:PD-infected plants. FIG. 6C (right panels) shows analogous results from plants infected with TRV:RU and TRV:BISCO carrying contiguous non-overlapping fragments of the rubisco cDNA. Plants infected with a TRV:RU vector only produced RU-siRNAs and plants infected with a TRV:BISCO vector only produced BISCO-siRNAs. Thus, taken together, these findings show that spreading of targeting does not occur with PDS and rubisco.

Thus simple VIGS of rubisco and PDS was different from that of GFP in that there was neither spreading nor RdDM (FIG. 5; (Jones et al., 1999; Thomas et al., 2001)) and because the continued presence of the initator viral RNA was required for persistence of silencing (Dalmay et al., 2001; Ruiz et al., 1998). Thus, these characteristics confirm the link between spreading and initiator-independent maintenance of silencing.

### Example 6

### Silencing Using Amplicon Constructs Based on Homology with PVX Genome.

The construct pGIIGPS carries the whole GUS gene, a fragment of sulphur gene and a fragment of the coat protein (CP) from PVX. Its construction is showed in FIG. 7. The cloning of the different fragments was made in a modified pGreenII 0229 vector (kindly provided by Dr. R. Hellens, JIC, Norwich, UK), which carries a gene that confers resistance to BASTA. The polylinker of pGreenII0229 was removed by digestion with SacI, blunt-ended and re-digested with Asp718. The polylinker was substituted by the 35S cassette from pJIT61 (kindly provided by P. Mullineaux, JIC, Norwich, UK), digested with EcoRV and Asp718 to produce the vector pGII61, which is now an expression vector. The GUS gene, containing an intron from ST-LS1 from potato, was amplified from plasmid pLaw3 using Expand Hi Fi DNA polymerase and the primers F5′GUS (5′TTATGTTACGTCCTGTAGAAACCC), and 3′GUS (5′TCATTGTTTGCCTCCCTGCTGC). The 2000 nt long PCR fragment was blunt ended and cloned into the HindIII blunt-ended site of pGII61 to produce the plasmid pGIIGUS. This plasmid was then digested with EcoRI and blunt ended using T4 DNA polymerase and used to clone a fragment of 742 nt from PVX CP generated by digestion of pgR106 (vector for PVX, kindly provided by Lu Rui, The Sainsbury Laboratory, Norwich, UK) with SalI and XhoI and blunt ended, producing the plasmid pGIIGP. This plasmid has a polylinker with sites for XbaI, BamHI, SmaI, XmaI and SacI where target genes can be cloned. As a target gene for spreading we introduced a fragment of sulphur gene from Arabidopsis (Kjemtrup et al., 1998). A 965 bp PCR fragment was amplified using Expand Hi Fi DNA polymerase (Boehringer) and the primers Sul1 (5′ccttggcgcgCCTTCACTCTCTTCTCCTTCC) and Sul2 (5′ccccttaattAATCTGGTCTTGAAGCTTGTCC) where the sequence in upper case corresponds to sulphur and the sequences in lower case introduce restriction site for AscI (Sul1) or PacI (Sul2). The fragment was blunt-ended and cloned into the Sac I, blunt ended site of pGIIGP, producing the plasmid pGIIGPS. The sulphur gene encodes for a magnesium chelatase involved in chlorophyll production. As a negative control, we made the same construction without the PVX CP fragment, which should not cause a silencing phenotype (pGIIGS, FIG. 8). This construct had the same sulphur fragment cloned into the EcoRI blunt-ended site of pGIIGUS.

Since these constructs were to be transformed into PVX/GFP amplicon Arabidopsis plants, which were already resistant to BASTA, the final constructs were moved to the vector pGreenII0029, which carried a gene for resistance to kanamycin in the T-DNA. A PCR fragment including the 35S promoter, GUS-IVS gene, Sulphur and PVX CP and 35S terminator was amplified from pGIIGPS and pGIIGS using the primers TR35S (5′ ccatatgtttaaaCCCCTACTCCAAAAATGTC) where the sequence in upper case corresponds to the 35S promoter and TR35T (5′ cccgtagtttaaacgtcgaggatATCGCATGC) where the sequences in upper case correspond to 35S terminator. The sequences in lower case in both primers include sites for PmeI restriction enzyme. The corresponding PCR fragments were digested with PmeI and cloned into pGreenII0029 to produce the final plasmids pKIIGPS as spreading construct and pKIIGS as non spreading negative control. Construction of pKIIGPS is shown in FIG. 9.

Results

PVX/GFP amplicon Arabidopsis plants were transformed either with pKIIGPS or pKIIGS constructs. As sulphur gene encodes for a magnesium chelatase involved in chlorophyll production, its silencing should produce total or partial yellow plants. Seeds from primary transformation were germinated in plates with GM medium supplemented with 500 μg/ml Carbenicillin, 200 μg/ml Augmentin and 50 μg/ml Kanamycin. Several Kan resistant transformant plants were obtained for both constructs, but no yellow silencing seedlings were obtained for construct pKIIGPS. Since it was believed that Kanamycin may have been playing a role against the silenced plants, which already “suffered” because of the silencing of the sulphur gene, we plated the seeds in GM plates with no Kan selection and with sucrose as a carbon source supply to bypass the lack of chlorophyll. After about 10 days, some yellow seedlings were observed among a lawn of green non transformed plants, showing that spreading had occurred. Those plants were pricked out to new GM/sucrose plates where were kept to maximise the possibility of survival. Tests can be done to show that they carry the spreading transgene. As a negative control, C-24 wt Arabidopsis plants (the background ecotype for PVX/GFP amplicon plants) were transformed with construct pKIIGPS. Seeds from primary transformants germinated in the same conditions did not produce yellow seedlings.

### Example 7

### Silencing using Amplicon Constructs Based on Homology with GFP.

PVX/GFP amplicon and C-24 plants were also transformed with constructs based on GFP homology as a trigger for spreading. Seeds from transformed PVX/GFP amplicon plants were germinated in GM plates under Kan selection and, after one week, small white (for PDS-carrying constructs) or yellow (for sulphur-carrying constructs) seedlings were visible. Nevertheless, these plants were not viable and died before producing the first true leaves. Seeds were then plated in GM medium without Kan selection and in the presence of sucrose. In addition, plates carrying the PDS constructs were put under dim light, to prevent excess of photobleaching. After 4 weeks in growth room there were many tiny white plants in the PDS plates and some yellow plants in the sulphur plates. There were no yellow or white plants in control plates with original PVX amplicon seeds. White plants from PDS plates were pricked out to new plates. They had white cotyledons and, some of them, green tiny first true leaves. Plants were kept in the growth room under the same conditions and during this time most of them developed green leaves and shoots. Some developed white sectors and a very few stayed almost completely white and very small. PCR to amplify GFP showed that the three types of plants had acquired the spreading PDS transgene. Control plants that never showed white colour did not have the transgene either. The fact that few plants were white and most of them turned green could be accounted for by excessively strong silencing that prevented survival of the plants. The same is true for the sulphur constructs.

### Example 8

### Silencing Using IR Constructs Based on Homology with GFP.

As an alternative to virus-based constructs (e.g. Amplicons), it may be preferred to use as an inducer a hairpin construct carrying an inverted repeat of a fragment of the transgene e.g. GFP gene. The “spreading constructs” can be the same as the GFP/PDS and GFP/sulphur ones used in Examples 6 and 7.

Both hairpin and spreading constructs may be put under a DEX promoter inducible by dexametasone, as well as under the 35S promoter.

Inducer constructs were built as follows:

As a vector for the construction of the hairpin, we used pFGC5941 (ChromDB, Arizona), which confers resistance to BASTA and drives transcription from 35S promoter, so that expression will be constitutive. A 686 nt fragment of GFP was amplified using primers GFP-1(X/A) (5′ aattcccgggcgcgccATGAAAGGAGAAGAACTTT), where the sequence in upper case corresponds to GFP and the sequence in lower case includes sites for XmaI and AscI restriction enzymes, and primer GFP-4(X/S) (5′ atattctagatttaaaTTCCGTCCTCCTTGAAAT), where the sequence in upper case corresponds to GFP and the one in lower case includes sites for XbaI and SwaI restriction enzymes. The amplified fragment (GF) was digested with SwaI and AscI and introduced into pFGC5941 previously digested with the same enzymes, to produce pssGF1. The same PCR fragment was then digested with XbaI and XmaI and introduced into pssGF1 previously digested with the same enzymes, to produce pGF/FG, where the GFP fragments are in inverted orientation respect to each other and separated by an intron already present in the original pFGC5941 vector (FIG. 10).

To transfer the hairpin to a vector with an inducible promoter, pGF/FG was digested with XmaI, blunt ended with T4 DNA polymerase, and redigested with XhoI, releasing the hairpin (GF-intron-FG). The hairpin was then ligated into pTA 231 vector (kindly provided by Prof. N. H. Chua, Rockefeller Univ, New York, USA) previously digested with PacI, blunt-ended with T4 DNA polymerase and redigested with XhoI to produce pTAdsGF, where the GF hairpin is under the influence of a promoter inducible by dexametasone (FIG. 11). As a negative control, the fragment GF-intron from pssGF1 was introduced into pTA231 in the same way, generating the construct pTAssGF, which should not produce any silencing.

Constructs pTAssGF and pTAdsGF were transformed into C-24, GFP and GFP/sde1 mutant plants. Seeds from primary transformation of GFP plants were germinated and selected in the presence of BASTA. Of 20 lines, 14 were green, indicating that in these lines the inducible promoter remains inactive before induction. Appropriate hairpin inducible lines are selected as a basis for subsequent transformations with different spreading constructs and to create a high thoughput system, using a cDNA library cloned into a GFP gene, for transformation and silencing any gene in Arabidopsis.

### Example 9

### Gene Silencing of osga and wx Genes by “Spreading” in Rice

Binary Vectors and Agrobacterium Strains

Binary vectors and Agrobacterium strains used for rice transformation are the following:

1^{st} round of transformation:

pGF-FG: LB-MASt::bar::MASp-CaMV35Sp::GF-ChsAintl::FG::OCSt-RB (based upon pFGC5941 vector system)

2^{nd} round of transformation:

pRT104, pRT105 and pRT106 were constructed by inserting PCR products into the ndeI site of the gfp gene of pGVT1 (made available by V. Thole, John Innes Centre, UK).

pGVT1=LB-NOSp::nptII::NOSt-CaMV35Sp::gfp::St-RB (pGreen-based vector)

pRT104: a 356 nt fragment of the osga gene was amplified by from rice wt genomic DNA (Nipponbare) and inserted in sense orientation into the ndeI site of the gfp gene of pGVT1.

pRT105: a 356 nt fragment of the osga gene was amplified by from rice wt genomic DNA (Nipponbare) and inserted in antisense orientation into the ndeI site of the gfp gene of pGVT1.

pRT106: a 639 nt fragment of the wx gene was amplified by from rice wt genomic DNA (Nipponbare) and inserted in sense orientation into the ndeI site of the gfp gene of pGVT1.

Plasmids were transformed into E. coli strain DH5 using the PEG-transformation technique and into Agrobacterium strains LBA4404 and GM3101 using a freeze-thaw technique. Agrobacterium strain harboured the following plasmids
Strain # 38: GM3101 containing pGF-FG
Strain # 42: LBA4404 containing pRT104 (pGreen-based) and pSa-Rep (pSoup-based).
Strain # 44: LBA4404 containing pRT105 (pGreen-based) and pSa-Rep (pSoup-based).
Strain # 45: LBA4404 containing pRT106 (pGreen-based) and pSa-Rep (pSoup-based).
   Rice Transformation Procedures

Mature seeds of rice (Oryza sativa L.) variety Nipponbare were used for callus production. Dehusked seeds were sterilised with half strength commercial bleach for 15 min and rinsed three times with sterile distilled water. The embryos were aseptically removed under a dissecting microscope and plated onto NBm medium (macro-element N6, micro-elements B5, Fe-EDTA, 30 g l⁻¹ sucrose, 2 mg l⁻¹ 2,4-D, 300 mg l⁻¹ casein hydrolysate, 500 mg l⁻¹ L-glutamine, 500 mg l⁻¹ L-proline, 2.5 g l⁻¹ Phytagel, pH 5.8, filter-sterilized vitamins B5 added after autoclavage) for 3 weeks in the dark at 25° C. Loose embryogenic transluscent globules (U), around 1 mm in size, were separated by rolling the callus grown from the original embryo onto the gelling agent. Globules were cultured for an additional 10 days onto fresh NBm medium (˜100 globules per plate) to produce embryogenic nodular units (ENU, Bec et. al. 1998), used as targets for transformation.

Agrobacterium strains were grown for 2 d at 28° C. on solid MG/L medium (Garfinkel and Nester 1980) supplemented by 200 M acetosyringone, 50 mg l⁻¹ Kanamycin (selection for pGreen- and pFGC5941-based vectors) and 10 mg l⁻¹ tetracyclin (selection for pSoup-based vectors). Bacteria cells were scooped up from the plate, re-suspended in 20 ml of SU4 liquid medium (macro-element N6, micro-elements MS, Fe-EDTA, 10 g l⁻¹ sucrose, 10 g l⁻¹ mannitol, pH 5.5, without antibiotics) and shaken for 1 hour at 28° C. Culture plates containing ENUs were flooded with bacterial suspension OD=1 (600 nm) for 5 min. Liquid was removed and each ENU was picked and blotted onto sterile filter paper before being placed onto co-cultivation medium (NBm medium supplemented by 200 M acetosyringone) for 2 days in the dark at 25° C. After co-culture, ENUs were put onto selection medium (NBm medium containing 150 mg l⁻¹ timentin plus either 5 mg l⁻¹ phosphinotrycin (PPT, selection using bar gene) or 100 mg l⁻¹ Geneticin (selection using nptII gene) in the dark at 25° C. L-glutamine was removed from all culture media when PPT was included. After two weeks culture, each callus (grown from an individual ENU) was split into 2 to 5 pieces. Pieces of callus were cultured for 3 additional weeks onto fresh NBm-based selection medium. The resistant calli grown from individual ENU, after 2+3 weeks selection, were or kept separated according to the separation undertaken at 2 weeks.

Transformed plants were regenerated from resistant calli using culture media all supplemented with 50 mg l⁻¹ Timentin and containing either 5 mg l⁻¹ phosphinotrycin (PPT, selection using bar gene) or 100 mg l⁻¹ Geneticin (selection using nptII gene). The resistant calli were transferred to PRm pre-regeneration medium (NBm medium without 2,4-D but with 2 mg l⁻¹ BAP, 1 mg l⁻¹ NAA, 5 mg l⁻¹ ABA) for 9 days in the dark at 28° C. Calli showing clear differential growth were then transferred to regeneration medium RNm (NBm medium without 2,4-D but with 3 mg l⁻¹ BAP, 0.5 mg l⁻¹ NAA) for 2-3 weeks in the light at 28° C. Only one plant was regenerated from each orignal ENU to guarantee that each plant represented an independent transformation event. Plants were developed on MSR6 medium (Vain et al. 1998) for 2-3 weeks at 28° C. in the light. Transformed plants were transferred to a controlled environment room for growth to maturity. All transgenic plants produced were used in further experiments to ensure the study of randomised independent transformation events with the widest spectrum of expression for the non-selected genes.

Genotyping and Phenotyping

Transformed rice plants from the 1^{st} round of transformation containing the transgenes and expressing small GF RNAs (20-30 nt) were selected using PCR analysis and Northern blots respectively. Two types of transgenic plant lines were identified: High and low production of small RNAs. T₁ seeds were obtained by self-pollination of primary transformed rice (T₀) plants. Analysis of plants from the 2^{nd} round of transformation may be used to demonstrate a slender phenotype for osga silencing and no iodine staining of pollen and embryos for wx silencing.

### REFERENCES

Inasmuch as they may be required or desired by the person skilled in the art to practice the present invention, all citations are specifically included herein by cross-reference.
Al-Kaff, N. S., Covey, S. N., Kreike, M. M., Page, A. M., Pinder, R., and Dale, P. J. (1998). Transcriptional and post-transcriptional plant gene silencing in response to a pathogen. Science 279, 2113-2115.
Aravin, A. A., Naumova, N. M., Tulin, A. V., Vagin, V. V., Rozovsky, Y. M., and Gvozdev, V. A. (2001). Double-stranded RNA-mediated silencing of genomic tandem repeats and transposable elements in the D-melanogaster germline. Current Biology 11, 1017-1027.
Bernstein, E., Caudy, A. A., Hammond, S. M., and Hannon, G. J. (2001). Role for a bidentate ribonuclease in the initiation step of RNA interference. Nature 409, 363-366.
Carthew, R. W. (2001). Gene silencing by double-stranded RNA. Current Opinion in Cell Biology 13, 244-248.
Chuang, C.-H., and Meyerowitz, E. M. (2000). Specific and heritable genetic interference by double-stranded RNA in Arabidopsis thaliana. Proc. Natl. Acad. Sci. USA 97, 4985-4990.
Cogoni, C., Irelan, J. T., Schumacher, M., Schmidhauser, T. J., Selker, E. U., and Macino, G. (1996). Transgene silencing of the Al-l gene in vegetative cells of Neurospora is mediated by a cytoplasmic effector and does not depend on DNA—DNA interactions or DNA methylation. EMBO J. 15, 3153-3163.
Cogoni, C., and Macino, G. (1999). Gene silencing in Neurospora crassa requires a protein homologous to RNA-dependent RNA polymerase. Nature 399, 166-169.
Cogoni, C., and Macino, G. (2000). Post-transcriptional gene silencing across kingdoms. Current Opinion in Genetics & Development 10, 638-643.
Covey, S. N., Al-Kaff, N. S., Langara, A., and Turner, D. S. (1997). Plants combat infection by gene silencing. Nature 385, 781-782.
Dalmay, T., Hamilton, A. J., Mueller, E., and Baulcombe, D. C. (2000a). Potato Virus X amplicons in arabidopsis mediate genetic and epigenetic gene silencing. Plant Cell 12, 369-379.
Dalmay, T., Hamilton, A. J., Rudd, S., Angell, S., and Baulcombe, D. C. (2000b). An RNA-dependent RNA polymerase gene in Arabidopsis is required for posttranscriptional gene silencing mediated by a transgene but not by a virus. Cell 101, 543-553.
Dalmay, T. D., Horsefield, R., Braunstein, T. H., and Baulcombe, D. C. (2001). SDE3 encodes an RNA helicase required for post-transcriptional gene silencing in Arabidopsis. EMBO Journal. 20, 2069-2078.
Demmig-Adams, B., and Adams, W. W. (1992). Photoprotection and other responses of plants to high light stress. Annu. Rev. Plant Physiol. Plant Mol. Biol. 43, 599-626.
Elbashir, S. M., Lendeckel, W., and Tuschl, T. (2001). RNA interference is mediated by 21- and 22-nucleotide RNAs. Genes & Development 15, 188-200.
Elmayan, T., and Vaucheret, H. (1996). Single copies of a 35S-driven transgene can undergo post-transcriptional silencing at each generation or can be transcriptionally inactivated in trans by a 35S silencer. Plant J. 9, 787-797.
English, J. J., Mueller, E., and Baulcombe, D. C. (1996). Suppression of virus accumulation in transgenic plants exhibiting silencing of nuclear genes. Plant Cell 8, 179-188.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-811.
Grishok, A., Tabara, H., and Mello, C. (2000). Genetic requirements for inheritance of RNAi in C. elegans. Science 287, 2494-2497.
Hamilton, A. J., and Baulcombe, D. C. (1999). A novel species of small antisense RNA in post-transcriptional gene silencing. Science 286, 950-952.
Hammond, S. M., Bernstein, E., Beach, D., and Hannon, G. (2000). An RNA-directed nuclease mediates post-transcriptional gene silencing in Drosophila cell extracts. Nature 404, 293-296.
Horsch, R. B., Fry, J. E., Hoffmann, N. L., Eichholtz, D., Rogers, S. G., and Fraley, R. T. (1985). A simple and general method of transferring genes into plants. Science 227, 1229-1231.
Hutvagner, G., McLachlan, J., Pasquinelli, A. E., Balint, E., Tuschl, T., and Zamore, P. D. (2001). A cellular function for the RNA-interference enzyme Dicer in the maturation of the let-7 small temporal RNA. Science 293, 834-838.
Jensen, S., Gassama, M. P., and Heidmann, T. (1999). Taming of transposable elements by homology-dependent gene silencing. Nature Genetics 21, 209-212.
Jones, L., Hamilton, A. J., Voinnet, O., Thomas, C. L., Maule, A. J., and Baulcombe, D. C. (1999). RNA-DNA interactions and DNA methylation in post-transcriptional gene silencing. Plant Cell 11, 2291-2302.
Jones, L., Ratcliff, F., and Baulcombe, D. C. (2001). RNA-directed transcriptional gene silencing in plants can be inherited independently of the RNA trigger and requires Met1 for maintenance. Current Biology 11, 747-757.
Jones, P. A., and Takai, D. (2001). The role of DNA methylation in mammalian epigenetics. Science 293, 1068-1070.
Ketting, R. F., and Plasterk, R. H. A. (2000). A genetic link between co-suppression and RNA interference in C. elegans. Nature 404, 296-298.
Kumagai, M. H., Donson, J., Della-Cioppa, G., Harvey, D., Hanley, K., and Grill, L. K. (1995). Cytoplasmic inhibition of carotenoid biosynthesis with virus-derived RNA. Proceedings Of The National Academy Of Sciences Of The United States Of America 92, 1679-1683.
Lindbo, J. A., Silva-Rosales, L., Proebsting, W. M., and Dougherty, W. G. (1993). Induction of a highly specific antiviral state in transgenic plants: implications for regulation of gene expression and virus resistance. Plant Cell 5, 1749-1759.
Matzke, M. A., Matzke, A. J. M., Pruss, G. J., and Vance, V. B. (2001). RNA-based silencing strategies in plants. Current Opinion in Genetics & Development 11, 221-227.
Morel, J.-B., Mourrain, P., Beclin, C., and Vaucheret, H. (2000). DNA methylation and chromatin structure affect transcriptional and post-transcriptional transgene silencing in Arabidopsis. Current Biology 10, 1591-1594.
Mourrain, P., Beclin, C., Elmayan, T., Feuerbach, F., Godon, C., Morel, J.-B., Jouette, D., Lacombe, A.-M., Nikic, S., Picault, N., Remoue, K., Sanial, M., Vo, T.-A., and Vaucheret, H. (2000). Arabidopsis SGS2 and SGS3 genes are required for posttranscriptional gene silencing and natural virus resistance. Cell 101, 533-542.
Pal-Bhadra, M., Bhadra, U., and Birchler, J. A. (1999). Cosuppression of nonhomologous transgenes in Drosophila involves mutually related endogenous sequences. Cell 99, 35-46.
Palauqui, J.-C., and Balzergue, S. (1999). Activation of systematic acquired silencing by localised introduction of DNA. Current Biology 9, 59-66.
Palauqui, J.-C., Elmayan, T., Pollien, J.-M., and Vaucheret, H. (1997). Systemic acquired silencing: transgene-specific post-transcriptional silencing is transmitted by grafting from silenced stocks to non-silenced scions. EMBO Journal. 16, 4738-4745.
Ratcliff, F., Harrison, B. D., and Baulcombe, D. C. (1997). A similarity between viral defense and gene silencing in plants. Science 276, 1558-1560.
Ratcliff, F., MacFarlane, S., and Baulcombe, D. C. (1999). Gene silencing without DNA: RNA-mediated cross protection between viruses. Plant Cell 11, 1207-1215.
Ratcliff, F., Martin-Hernandez, A. M., and Baulcombe, D. C. (2001). Tobacco rattle virus as a vector for analysis of gene function by silencing. Plant Journal 25, 237-245.
Ruiz, M. T., Voinnet, O., and Baulcombe, D. C. (1998). Initiation and maintenance of virus-induced gene silencing. Plant Cell 10, 937-946.
Schiebel, W., Haas, B., Marinkovic, S., Klanner, A., and Sanger, H. L. (1993a). RNA-directed RNA polymerase from tomato leaves. I. Purification and physical properties. The Journal of Biological Chemistry. 263, 11851-11857.
Schiebel, W., Haas, B., Marinkovic, S., Klanner, A., and Sanger, H. L. (1993b). RNA-directed RNA polymerase from tomato leaves. II. Catalytic in vitro properties. The Journal of Biological Chemistry. 268, 11858-11867.
Smardon, A., Spoerke, J. M., Stacey, S. C., Klein, M. E., Mackin, N., and Maine, E. M. (2000). EGO-1 is related to RNA-directed RNA polymerase and functions in germ-line development and RNA interference in C. elegans. Curr. Biol. 10, 169-178.
Smith, N. A., Singh, S. P., Wang, M. B., Stoutjesdijk, P. A., Green, A. G., and Waterhouse, P. M. (2000). Gene expression—Total silencing by intron-spliced hairpin RNAs. Nature 407, 319-320.
Thomas, C. L., Jones, L., Baulcombe, D. C., and Maule, A. J. (2001). Size constraints for targeting post-transcriptional gene silencing and for RNA-directed methylation in Nicotiana benthamiana using a potato virus X vector. Plant Journal 25, 1-11.
Voinnet, O., and Baulcombe, D. C. (1997). Systemic signalling in gene silencing. Nature 389, 553.
Voinnet, O., Vain, P., Angell, S., and Baulcombe, D. C. (1998). Systemic spread of sequence-specific transgene RNA degradation is initiated by localised introduction of ectopic promoterless DNA. Cell 95, 177-187.
Wang, M. B., Wesley, S. V., Finnegan, E. J., Smith, N. A., and Waterhouse, P. M. (2001). Replicating satellite RNA induces sequence-specific DNA methylation and truncated transcripts in plants. RNA-a Publication of the RNA Society 7, 16-28.
Wassenegger, M. (2000). RNA-directed DNA methylation. Plant Molecular Biology 43, 203-220.
Waterhouse, P. M., Wang, M. B., and Lough, T. (2001). Gene silencing as an adaptive defence against viruses. Nature 411, 834-842.
Wu-Scharf, D., Jeong, B.-r., Zhang, C., and Cerutti, H. (2000). Transgene and transposon silencing in Chlamydomonas reinhardtii by a DEAH-Box RNA helicase. Science 290, 1159-1162.
Zamore, P. D., Tuschl, T., Sharp, P. A., and Bartel, D. P. (2000). RNAi: Double-stranded RNA directs the ATP-dependent cleavage of mRNA at 21 to 23 nucleotide intervals. Cell 101, 25-33.

### ADDITIONAL REFERENCES

Bec S, Chen L, Ferriere N M, Legave T, Fauquet C, Guideroni E (1998) Comparitive histology of microprojectile-mediated gene transfer to embryonic calli in japonica rice (oryza sativa L.): influence of the structural organization of target tissues on genotype transformation ability. Plant Science 138:177-190
Chuang, C.-H., and Meyerowitz, E. M. (2000). Specific and heritable genetic interference by double-stranded RNA in Arabidopsis thaliana. Proc. Natl. Acad. Sci. USA 97, 4985-4990.
Garfinkel M, Nester EW (1980) Agrobacterium tumefaciens mutants affected in crown gall tumorigenesis and octopine catabolism. Journal of Bacteriology 144:732-743
Kjemtrup, S., Sampson, K. S., Peele, C. G., Nguyen, L. V., Conkling, M. A., Thompson, W. F., and Robertson, D. (1998). Gene silencing from plant DNA carried by a geminivirus. Plant J. 14, 91-100.
Kumagai, M. H., Donson, J., Della-Cioppa, G., Harvey, D., Hanley, K., and Grill, L. K. (1995). Cytoplasmic inhibition of carotenoid biosynthesis with virus-derived RNA. Proc. Natl. Acad. Sci. USA 92, 1679-1683.
Peele, C., Jordan, C. V., Muangsan, N., Turnage, M., Egelkrout, E., Eagle, P., Hanley-Bowdoin, L., and Robertson, D. (2001). Silencing of a meristematic gene using geminivirus-derived vectors. Plant J. 27, 357-366.
Ratcliff, F., Martin-Hernandez, A. M., and Baulcombe, D. C. (2001). Tobacco rattle virus as a vector for analysis of gene function by silencing. Plant J. 25, 237-245.
Ruiz, M. T., Voinnet, O., and Baulcombe, D. C. (1998). Initiation and maintenance of virus-induced gene silencing. Plant Cell 10, 937-946.
Smith, N. A., Singh, S. P., Wang, M. B., Stoutjesdijk, P. A., Green, A. G., and Waterhouse, P. M. (2000). Gene expression—Total silencing by intron-spliced hairpin RNAs. Nature 407, 319-320.
Vain P, Worland B, Clarke M C, Richard G, Beavis M, Liu H, Kohli A, Leech M, Snape J W, Christou P, Atkinson H (1998) Expression of an engineered proteinase inhibitor (Oryzacystatin-IΔd86) for nematode resistance in transgenic rice plants. Theor and Appl Genet 96:266-271
Wesley, S. V., Helliwell, C. A., Smith, N. A., Wang, M., Rouse, D. T., Liu, Q., Gooding, P. S., Singh, S. P., Abbott, D., Stoutjesdijk, P. A., Robinson, S. P., Gleave, A. P., Green, A. G., and Waterhouse, P. (2001). Construct design for efficient, effective and high-throughput gene silencing in plants. Plant J. 27, 581-590.

### SEQUENCE APPENDIX

Key to sequence annotation;

| | |
|---|---|
| Upper-case | plasmid backbone sequence |
| Lower-case | CaMV 35S promoter sequence |
| UPPER-CASE UNDERLINED | GUS-IVS |
| UPPER-CASE AND BOLD | PVX sequence |
| Lower-case and bold | Sulphur sequence |
| Lower-case italics | CaMV 35S terminator sequence |
| Lower-case, bold and | GFP sequences |
| Underlined | |
| UPPER CASE ITALICS | CSHA intron sequence |
| Lower case underlined | OCS 3′ terminator sequence |
| Lower case underlined | Inducible elements and BASTA |
| and italics | resistance in pTAdsGF |
| UPPER CASE, ITALICS | Pea Rubisco small subunit gene |
| UNDERLINED | terminator |

1-Sequence of pKIIGPS.

TCTTGGCAGGATATATTGTGGTGTAACGTTATCAGCTTGCATGCCGGTCGATCTAGTAACATAGATGACACCGC
GCGCGATAATTTATCCTAGTTTGCGCGCTATATTTTGTTTTCTATCGCGTATTAAATGTATAATTGCGGGACTC
TAATCAAAAAACCCATCTCATAAATAACGTCATGCATTACATGTTAATTATTACATGCTTAACGTAATTCAACA
GAAATTATATGATAATCATCGCAAGACCGGCAACAGGATTCAATCTTAAGAAACTTTATTGCCAAATGTTTGAA
CGATCTGCTTGACTCTAGCTAGAGTCCGAACCCCAGAGTCCCGCTCAGAAGAACTCGTCAAGAAGGCGATAGAA
GGCGATGCGCTGCGAATCGGGAGCGGCGATACCGTAAAGCACGAGGAAGCGGTCAGCCCATTCGCCGCCAAGCT
CTTCAGCAATATCACGGGTAGCCAACGCTATGTCCTGATAGCGGTCCGCCACACCCAGCCGGCCACAGTCGATG
AATCCAGAAAAGCGGCCATTTTCCACCATGATATTCGGCAAGCAGGCATCGCCCTGGGTCACGACGAGATCCTC
GCCGTCGGGCATCCGCGCCTTGAGCCTGGCGAACAGTTCGGCTGGCGCGAGCCCCTGATGCTCTTCGTCCAGAT
CATCCTGATCGACAAGACCGGCTTCCATCCGAGTACGTCCTCGCTCGATGCGATGTTTCGCTTGGTGGTCGAAT
GGGCAGGTAGCCGGATCAAGCGTATGCAGCCGCCGCATTGCATCAGCCATGATGGATACTTTCTCGGCAGGAGC
AAGGTGAGATGACAGGAGATCCTGCCCCGGCACTTCGCCCAATAGCAGCCAGTCCCTTCCCGCTTCAGTGACAA
CGTCGAGCACAGCTGCGCAAGGAACGCCCGTCGTGGCCAGCCACGATAGCCGCGCTGCCTCGTCTTGGAGTT
CATTCAGGGCACCGGACAGGTCGGTCTTGACAAAAAGAACCGGGCGCCCCTGCGCTGACAGCCGGAACACGGCG
GCATCAGAGCAGCCGATTGTCTGTTGTGCCCAGTCATAGCCGAATAGCCTCTCCACCCAAGCGGCCGGAGAACC
TGCGTGCAATCCATCTTGTTCAATCATGCCTCGATCGAGTTGAGAGTGAATATGAGACTCTAATTGGATACCGA
GGGGAATTTATGGAACGTCAGTGGAGCATTTTTGACAAGAAATATTTGCTAGCTGATAGTGACCTTAGGCGACT
TTTGAACGCGCAATAATGGTTTCTGACGTATGTGCTTAGCTCATTAAACTCCAGAAACCCGCGGCTGAGTGGCT
CCTTCAACGTTGCGGTTCTGTCAGTTCCAAACGTAAAACGGCTTGTCCCGCGTCATCGGCGGGGGTCATAACGT
GACTCCCTTAATTCTCATGTATCGATAACATTAACG
TTTACAATTTCGCGCCATTCGCCATTCAGGCTGCGCAACTGTTGGGAAGGGCGATCGGTGCGGGCCTCTTCGCT
ATTACGCCAGCTGGCGAAAGGGGGATGTGCTGCAAGGCGATTAAGTTGGGTAACGCCAGGGTTTTCCCAGTCAC
GACGTTGTAAAACGACGGCCAGTGAATTGTAATACGACTCACTATAGGGCGAATTGggtacccccctactccaa
aaatgtcaaagatacagtctcagaagaccaaagggctattgagacttttcaacaaagggtaatttcgggaaacc
tcctc
Ggattccattgcccagctatctgtcacttcatcgaaaggacagtagaaaaggaaggtggctcctacaaatgcca
tcattgcga
Taaaggaaaggctatcattcaagatgcctctgccgacagtggtcccaaagatggacccccacccacgaggagca
tcgtgga
Aaaagaagacgttccaaccacgtcttcaaagcaagtggattgatgtgacatctccactgacgtaagggatgacg
cacaatccc
ActatccttcgcaagacccttcctctatataaggaagttcatttcatttggagaggacagcccaagctTTATGT
TACGT
CCTGTAGAAACCCCAACCCGTGAAATCAAAAAACTCGACGGCCTGTGGGCATTCAGTCTGGATCGCGAAAACTG
TGGAATTGATCAGCGTTGGTGGGAAAGCGCGTTACAAGAAAGCCGGGCAATTGCTGTGCCAGGCAGTTTTAACG
ATCAGTTCGCCGATGCAGATATTCGTAATTATGCGGGCAACGTCTGGTATCAGCGCGAAGTCTTTATACCGAAA
GGTTGGGCAGGCCAGCGTATCGTGCTGCGTTTCGATGCGGTCACTCATTACGGCAAAGTGTGGGTCAATAATCA
GGAAGTGATGGAGCATCAGGGCGGCTATACGCCATTTGAAGCCGATGTCACGCCGTATGTTATTGCCGGGAAAA
GTGTACGTAAGTTTCTGCTTCTACCTTTGATATATATATAATAATTATCATTAATTAGTAGTAATATAATATTT
CAAATATTTTTTTCAAAATAAAAGAATGTAGTATATAGCAATTTTTCTGTAGTTTATAAGTGTGTATATTTTAA
TTTATAACTTTTCTAATATATGACCAAAATTTGTTGATGTGCAGGTATCACCGTTTGTGTGAACAACGAACTGA
ACTGGCAGACTATCCCGCCGGGAATGGTGATTACCGACGAAAACGGCAAGAAAAAGCAGTCTTACTTCCATGAT
TTCTTTAACTATGCCGGATCATCGCAGCGTAATGCTCTACACCACGCCGAACACCTGGGTGGACGATATCACCG
TGGTGACGCATGTCGCGCAAGACTGTAACCACGCGTCTGTTGACTGGCAGGTGGTGGCCAATGGTGATGTCAGC
GTTGAACTGCGTGATGCGGATCAACAGGTGGTTGCAACTGGACAAGGCACTAGCGGGACTTTGCCAAGTGGGAA
TCCGCACCTCTGGCAACCGGGTGAAGGTTATCTCTATGAACTGTGCGTCACAGCCAAAAGCCAGACAGAGTGTG
ATATCTACCCGCTTCGCGTCGGCATCCGGTCAGTGGCAGTGAAGGGCGAACAGTTCCTGATTAACCACAAACCG
TTCTACTTTACTGGCTTTGGTCGTCATGAAGATGCGGACTTACGTGGCAAAGGATTCGATAACGTGCTGATGGT
GCACGACCACGCATTAATGGACTGGATTGGGGCCAACTCCTACCGTACCTCGCATTACCCTTACGCTGAAGAGA
TGCTCGACTGGGCAGATGAACATGGCATCGTGGTGATTGATGAAACTGCTGCTGTCGGCTTTAACCTCTCTTTA
GGCATTGGTTTCGAAGCGGGCAACAAGCCGAAAGAACTGTACAGCGAAGAGGCAGTCAACGGGGAAACTCAGCA
AGCGCACTTACAGGCGATTAAAGAGCTGATAGCGCGTGACAAAAACCACCCAAGCGTGGTGATGTGGAGTATTG
CCAACGAACCGGATACCCGTCCGCAAGGTGCACGGGAATATTTCGCGCCACTGGCGGAAGCAACGCGTAAACTC
GACCCGACGCGTCCGATCACCTGCGTCAATGTAATGTTCTGCGACGCTCACACCGATACAATCAGCGATCTCTT
TGATGTGCTGTGCCTGAACCGTTATTACGGATGGTATGTCCAAAGCGGCGATTTGGAAACGGCAGAGAAGGTAC
TGGAAAAAGAACTTCTGGCCTGGCAGGAGAAACTGCATCAGCCGATTATCATCACCGAATACGGCGTGGATACG
TTAGCCGGGCTGCACTCAATGTACACCGACATGTGGAGTGAAGAGTATCAGTGTGCATGGCTGGATATGTATCA
CCGCGTCTTTGATCGCGTCAGCGCCGTCGTCGGTGAACAGGTATGGAATTTCGCCGATTTTGCGACCTCGCAAG
GCATATTGCGCGTTGGCGGTAACAAGAAAGGGATCTTCACTCGCGACCGCAAACCGAAGTCGGCGGCTTTTCTG
CTGCAAAAACGCTGGACTGGCATGAACTTCGGTGAAAAACCGCAGCAGGGAGGCAAACAATGAagctttctaga
ggatcccccggggccttggcgcgccttcactctcttctccttcctcaaaa
ccttcctcctcccccatttgcttcaggccaggtaaattgtttggaagcaagttaaatgcaggaatccaaataag
gcca
aagaagaacaggtctcgttaccatgtttcggttatgaatgtagccactgaaatcaactctactgaacaagtagt
aggg
aagtttgattcaaagaagagtgcgagaccggtttatccatttgcagctatagtagggcaagatgagatgaagtt
atgt
cttttgttgaatgttattgatccaaagattggtggtgttatgattatgggagatagaggaactggaaaatctac
aactg
ttagatcattagttgatctgttacctgagattaatgtagttgcaggtgacccgtataactcggatccgatagat
cctgag
tttatgggtgttgaagtaagagagagagttgagaaaggagagcaagttcctgttattgcgactaagattaatat
ggttg
atcttcctttgggtgcaacagaagatagagtttgtggaaccatcgatatcgaaaaggctttgacagaaggtgta
aaag
cctttgagcctggtttgttggctaaagctaatagagggattctttatgttgatgaagttaatctcttggatgat
catttggtt
gatgttcttttggattcagctgcttctggttggaatacggttgagagagaagggatttcgatttctcacccggc
gaggttta
tcttgatcggttcaggaaatccggaagaaggagagcttaggccacagcttcttgatcggtttggtatgcatgca
caagt
agggacggttagagatgctgatttacgggtcaagattgttgaagagagagctcgtttcgatagtaacccaaagg
atttc
cgtgacacttacaaaaccgagcaggacaagcttcaagaccagattaattaaggggcgaattTCGACCGCCGATA
AGCTTGATAGGGCCATTGCCGATCTCAAGCCACTCTCCGTTGAACGGTTAAGTTTCCATTGATACTCGAAAGAT
GTCAGCACCAGCTAGCACAACACAGCCCATAGGTCAACTACCTCAAACTACCACAAAAACTGCAGGCGCAACTC
CTGCCACAGCTTCAGGCCTGTTCACCATCCCGGATGGGGATTTCTTTAGTACAGCCCGTGCCATAGTAGCCAGC
AATGCTGTCGCAACAAATGAGGACCTCAGCAAGATTGAGGCTATTTGGAAGGACATGAAGGTGCCCACAGACAC
TATGGCACAGGCTGCTTGGGACTTAGTCAGACACTGTGCTGATGTAGGATCATCCGCTCAAACAGAAATGATAG
ATACAGGTCCCTATTCCAACGGCATCAGCAGAGCTAGACTGGCAGCAGCAATTAAAGAGGTGTGCACACTTAGG
CAAAATTTTGCATGAA
GTATGCTCCAGTGGTATGGAACTQGATGTTAACTAACAACAGTCCACCTGCTAACTGGCAAGCACAAGGTTTCA
AGCCTGAGCACAAATTCGCTGCATTCGACTTCTTCAATGGAGTCACCAACCCAGCTGCCATCATGCCCAAAGAG
GGGCTCATCCGG
CCACCGTCTGAAGCTGAATGAATGCTGCCCAAACTGCTGCCTTTGTGAAGATTACAAAGGCCAGGGCACAATCCA
ACGACTTTGCCAGCCTAGATGCAGCTGTCACTCGAaattcggtacgctgaaatcaccagtctctctctacaaatct
atctctctctattttctccataaa ta
atgtgtgagtagtttcccgataagggaaattagggttcttatagggtttcgctcatgtgttgagcatataagaaac
ccttagtatg
tatttgtatttgtaaaatacttctatcaataaaatttctaattcctaaaaccaaaatccagtactaaaatccagat
ctcctaaagtccc
tatagatctttgtcgtgaatataaaccagacacgagacgactaaacctggagcccagacgccgttcgaagctagaa
gtacc
gcttaggcaggaggccgttagggaaaagatgctaaggcagggttggttacgttgactcccccgtaggtttggttta
aatatga
tgaagtggacggaaggaaggaggaagacaaggaaggataaggttgcaggccctgtgcaaggtaagaagatggaaa
tttgatagaggtacgctactatacttatactatacgctaagggaatgcttgtatttataccctataccccctaata
accccttatca
atttaagaaataatccgcataagcccccgcttaaaaattggtatcagagccatgaataggtctatgaccaaaactc
aagag
gataaaacctcaccaaaatacgaaagagttcttaactctaaagataaaagatctttcaagatcaaaactagttccc
tcaca
ccggagcatgcgatccagcttttgTTCCCTTTAGTGAGGGTTAATTCCGAGCTTGGCGTAATCATGGTCATAGCTG
TTTCCTGTGTGAAATTGTTATCCGCTCACAATTCCACACAACATACGAGCCGGAAGCATAAAGTGTAAAGCCTGGG
GTGCCTAATGAGTGAGCTAACTCACATTAATTGCGTTGCGCTCACTGCCCGCTTTCCAGTCGGGAAACCTGTCGTG
CCAGCTGCATTAATGAATCGGCCAACGCGCGGGGAGAGGCGGTTTGCGTATTGGGCGCTCTTCCGCTTCCTCGCTC
ACTGACTCGCTGCGCTCGGTCGTTCGGCTGCGGCGAGCGGTATCAGCTCACTCAAAGGCGGTAATACGGTTATCCA
CAGAATCAGGGGATAACGCAGGAAAGAACATGAAGGCCTTGACAGGATATATTGGCGGGTAAACTAAGTCGCTGTA
TGTGTTTGTTTGAGATCTCATGTGAGCAAAAGGCCAGCAAAAGGCCAGGAACCGTAAAAAGGCCGCGTTGCTGGCG
TTTTTCCATAGGCTCCGCCCCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTGGTAACAGGATTAGCAGAGC
GAGGTATGTAGGCGGTGCTACAGAGTTCTTGAAGTGGTGGCCTAACTACGGCTACACTAGAAGAACAGTATTTGGT
ATCTGCGCTCTGCTGAAGCCAGTTACCTTCGGAAGAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGTGTAACATTGGTCTAGTG
ATTAGAAAAACTCATCGAGCATCAAATGAAACTGCAATTTATTCATATCAGGATTATCAATACCATATTTTTGAAA
AAGCCGTTTCTGTAATGAAGGAGAAAACTCACCGAGGCAGTTCCATAGGATGGCAAGATCCTGGTATCGGTCTGCG
ATTCCGACTCGTCCAACATCAATACAACCTATTAATTTCCCCTCGTCAAAAATAAGGTTATCAAGTGAGAAATCAC
CATGAGTGACGACTGAATCCGGTGAGAATGGCAAAAGTTTATGCATTTCTTTCCAGACTTGTTCAACAGGCCAGCC
ATTACGCTCGTCATCAAAATCACTCGCATCAACCAAACCGTTATTCATTCGTGATTGCGCCTGAGCGAGACGAAAT
ACGCGATCGCTGTTAAAAGGACAATTACAAACAGGAATCGAATGCAACCGGCGCAGGAACACTGCCAGCGCATCAA
CAATATTTTCACCTGAATCAGGATATTCTTCTAATACCTGGAATGCTGTTTTCCCTGGGATCGCAGTGGTGAGTAA
CCATGCATCATCAGGAGTACGGATAAAATGCTTGATGGTCGGAAGAGGCATAAATTCCGTCAGCCAGTTTAGTCTG
ACCATCTCATCTGTAACAACATTGGCAACGCTACCTTTGCCATGTTTCAGAAACAACTCTGGCGCATCGGGCTTCC
CATACAATCGGTAGATTGTCGCACCTGATTGCCCGACATTATCGCGAGCCCATTTATACCCATATAAATCAGCATC
CATGTTGGAATTTAATCGCGGCCTTGAGCAAGACGTTTCCCGTTGAATATGGCTCATAACACCCCTTGTATTACTG
TTTATGTAAGCAGACAGTTTTATTGTTCATGATGATATATTTTTATCTTGTGCAATGTAACATCAGAGATTTTGAG
ACACAACGTGGCTTTGTTGAATAAATCGAACTTTTGCTGAGTTGAAGGATCAGATCACGCATCTTCCCGACAACGC
AGAC
CGTTCCGTGGCAAAGCAAAAGTTCAAAATCACCAACTGGTCCACCTACAACAAAGCTCTCATCAACCGTGGCTC
CCTCACTTTCTGGCTGGATGATGGGGCGATTCAGGCGATCCCCATCCAACAGCCCGCCGTCGAGCGGGCTTTTT
TATCCCCGGAAG
CCTGTGGATAGAGGGTAGTTATCCACGTGAAACCGCTAATGCCCCGCAAAGCCTTGATTCACGGGGCTTTCCGG
CCCGCTCCAAAAACTATCCACGTGAAATCGCTAATCAGGGTACGTGAAATCGCTAATCGGAGTACGTGAAATCG
CTAATAAGGTCA
CGTGAAATCGCTAATCAAAAAGGCACGTGAGAACGCTAATAGCCCTTTCAGATCAACAGCTTGCAAACACCCCT
CGCTCCGGCAAGTAGTTACAGCAAGTAGTATGTTCAATTAGCTTTTCAATTATGAATATATATATCAATTATTG
GTCGCCCTTGGCTTGTGGACAATGCGCTACGCGCACCGGCTCCGCCCGTGGACAACCGCAAGCGGTTGCCCACC
GTCGAGCGCCAGCGCCTTTGCCCACAACCCGGCGGCCGGCCGCAACAGATCGTTTTATAAATTTTTTTTTTTGA
AAAAGAAAAAGCCCGAAAGGCGGC
AACCTCTCGGGCTTCTGGATTTCCGATCCCCGGAATTAGAGA

2-Sequence of pKIIGS. 35S Cassette with GUS and Sulphur Inserts.

ggtacccccctactccaaaaatgtcaaagatacagtctcagaagaccaaagggctattgagacttttcaacaaa
gggtaatttc
gggaaacctcctcggattccattgcccagctatctgtcacttcatcgaaaggacagtagaaaaggaaggtggct
cctacaaat
gccatcattgcgaTaaaggaaaggctatcattcaagatgcctctgccgacagtggtcccaaagatggaccccca
cccacga
ggagcatcgtggaaaaagaagacgttccaaccacgtcttcaaagcaagtggattgatgtgacatctccactgac
gtaaggg
atgacgcacaatcccActatccttcgcaagacccttcctctatataaggaagttcatttcatttggagaggaca
gcccaagctT
TATGTTACGTCCTGTAGAAACCCCAACCCGTGAAATCAAAAAACTCGACGGCCTGTGGGCATTCAGTCTGGATC
GCGAAAACTGTGGAATTGATCAGCGTTGGTGGGAAAGCGCGTTACAAGAAAGCCGGGCAATTGCTGTGCCAGGC
AGTTTTAACGATCAGTTCGCCGATGCAGATATTCGTAATTATGCGGGCAACGTCTGGTATCAGCGCGAAGTCTT
TATACCGAAAGGTTGGGCAGGCCAGCGTATCGTGCTGCGTTTCGATGCGGTCACTCATTACGGCAAAGTGTGGG
TCAATAATCAGGAAGTGATGGAGCATCAGGGCGGCTATACGCCATTTGAAGCCGATGTCACGCCGTATGTTATT
GCCGGGAAAAGTGTACGTAAGTTTCTGCTTCTACCTTTGATATATATATAATAATTATCATTAATTAGTAGTAA
TATAATATTTCAAATATTTTTTTCAAAATAAAAGAATGTAGTATATAGCAATTTTTCTGTAGTTTATAAGTGTG
TATATTTTAATTTATAACTTTTCTAATATATGACCAAAATTTGTTGATGTGCAGGTATCACCGTTTGTGTGAAC
AACGAACTGAACTGGCAGACTATCCCGCCGGGAATGGTGATTACCGACGAAAACGGCAAGAAAAAGCAGTCTTA
CTTCCATGATTTCTTTAACTATGCCGGATCATCGCAGCGTAATGCTCTACACCACGCCGAACACCTGGGTGGAC
GATATCACCGTGGTGACGCATGTCGCGCAAGACTGTAACCACGCGTCTGTTGACTGGCAGGTGGTGGCCAATGG
TGATGTCAGCGTTGAACTGCGTGATGCGGATCAACAGGTGGTTGCAACTGGACAAGGCACTAGCGGGACTTTGC
CAAGTGGGAATCCGCACCTCTGGCAACCGGGTGAAGGTTATCTCTATGAACTGTGCGTCACAGCCAAAAGCCAG
ACAGAGTGTGATATCTACCCGCTTCGCGTCGGCATCCGGTCAGTGGCAGTGAAGGGCGAACAGTTCCTGATTAA
CCACAAACCGTTCTACTTTACTGGCTTTGGTCGTCATGAAGATGCGGACTTACGTGGCAAAGGATTCGATAACG
TGCTGATGGTGCACGACCACGCATTAATGGACTGGATTGGGGCCAACTCCTACCGTACCTCGCATTACCCTTAC
GCTGAAGAGATGCTCGACTGGGCAGATGAACATGGCATCGTGGTGATTGATGAAACTGCTGCTGTCGGCTTTAA
CCTCTCTTTAGGCATTGGTTTCGAAGCGGGCAACAAGCCGAAAGAACTGTACAGCGAAGAGGCAGTCAACGGGG
AAACTCAGCAAGCGCACTTACAGGCGATTAAAGAGCTGATAGCGCGTGACAAAAACCACCCAAGCGTGGTGATG
TGGAGTATTGCCAACGAACCGGATACCCGTCCGCAAGGTGCACGGGAATATTTCGCGCCACTGGCGGAAGCAAC
GCGTAAACTCGACCCGACGCGTCCGATCACCTGCGTCAATGTAATGTTCTGCGACGCTCACACCGATACCATCA
GCGATCTCTTTGATGTGCTGTGCCTGAACCGTTATTACGGATGGTATGTCCAAAGCGGCGATTTGGAAACGGCA
GAGAAGGTACTGGAAAAAGAACTTCTGGCCTGGCAGGAGAAACTGCATCAGCCGATTATCATCACCGAATACGG
CGTGGATACGTTAGCCGGGCTGCACTCAATGTACACCGACATGTGGAGTGAAGAGTATCAGTGTGCATGGCTGG
ATATGTATCACCGCGTCTTTGATCGCGTCAGCGCCGTCGTCGGTGAACAGGTATGGAATTTCGCCGATTTTGCG
ACCTCGCAAGGCATATTGCGCGTTGGCGGTAACAAGAAAGGGATCTTCACTCGCGACCGCAAACCGAAGTCGGC
GGCTTTTCTGCTGCAAAAACGCTGGACTGGCATGAACTTCGGTGAAAAACCGCAGCAGGGAGGCAAACAATGAa
gctttctagaggatcccccggggcgaattccttggcgcgccttcaatctcttctccttcctcaaaa
ccttcctcctcccccatttgcttcaggccaggtaaattgtttggaagcaagttaaatgcaggaatccaaataag
gcca
aagaagaacaggtctcgttaccatgtttcggttatgaatgtagccactgaaatcaactctactgaacaagtagt
aggg
aagtttgattcaaagaagagtgcgagaccggtttatccatttgcagctatagtagggcaagatgagatgaagtt
atgt
cttttgttgaatgttattgatccaaagattggtggtgttatgattatgggagatagaggaactggaaaatctac
aactg
ttagatcattagttgatctgttacctgagattaatgtagttgcaggtgacccgtataactcggatccgatagat
cctgag
tttatgggtgttgaagtaagagagagagttgagaaaggagagcaagttcctgttattgcgactaagattaatat
ggttg
atcttcctttgggtgcaacagaagatagagtttgtggaaccatcgatatcgaaaaggctttgacagaaggtgta
aaag
cctttgagcctggtttgttggctaaagctaatagagggattctttatgttgatgaagttaatctcttggatgat
catttggtt
gatgttcttttggattcagctgcttctggttggaatacggttgagagagaagggatttcgatttctcacccggc
gaggttta
tcttgatcggttcaggaaatccggaagaaggagagcttaggccacagcttcttgatcggtttggtatgcatgca
caagt
agggacggttagagatgctgatttacgggtcaagattgttgaagagagagctcgtttcgatagtaacccaaagg
atttc
cgtgacacttacaaaaccgagcaggacaagcttcaagaccagattaattaaggggaattcggtacgctgaaatc
acca
gtctctctctacaaatctatctctctctattttctccataaataatgtgtgagtagtttcccgataagggaaat
tagggttcttatagg
gtttcgctcatgtgttgagcatataagaaacccttagtatgtatttgtatttgtaaaatacttctatcaataaa
atttctaattccta
aaaccaaaatccagtactaaaatccagatctcctaaagtccctatagatctttgtcgtgaatataaaccagaca
cgagac
gactaaacctggagcccagacgccgttcgaagctagaagtaccgcttaggcaggaggccgttagggaaaagatg
cta
aggcagggttggttacgttgactcccccgtaggtttggtttaaatatgatgaagtggacggaaggaaggaggaa
gacaa
ggaaggataaggttgcaggccctgtgcaaggtaagaagatggaaatttgatagaggtaagctactatacttata
ctatac
gctaagggaatgcttgtatttataccctataccccctaataaccccttatcaatttaagaaataatccgcataa
gcccccgc
ttaaaaattggtatcagagccatgaataggtctatgaccaaaactcaagaggataaaacctcaccaaaatacga
aaga
gttcttaactctaaagataaaagatcattcaagatcaaaactagttccctcacaccggagcatgcgatcaagct
tttgTTC

3-Sequence of pGF/FG. Showed from Promoter to Terminator.

tactccaaaaatgtcaaagatacagtctcagaagaccaaagggctattgagacttttcaacaaaggataatttc
gggaaa
cctcctcggattccattgcccagctatctgtcacttcatcgaaaggacagtagaaaaggaaggtggctcctaca
aatgcc
atcattgcgataaaggaaaggctatcattcaagatctgcctctgccgacagtggtcccaaagatggacccccac
ccacga
ggagcatcgtggaaaaagaagacgttccaaccacgtcttcaaagcaagtggattgatgtgacatctccactgac
gtaagg
gatgacgcacaatcccactatccttcgcaagacccttcctctatataaggaagttcatttcatttggagaggac
acgctc
gagtataagagctcatttttacaacaattaccaacaacaacaaacaacaaacaacattacaattacatttacaa
ttatcc
atGGCGCGCCagtaaaggagaagaacttttcactggagttgtcccaattcttgttgaattagatggtgatgtt
aatgggcacaaattttctgtcagtggagagggtgaaggtgatgcaacatacggaaaacttacccttaaatttat
t
tgcactactggaaaactacctgttccatggccaacacttgtcactactttctcttatggtgttcaatgcttttc
aagat
acccagatcatatgaagcggcacgacttcttcaagagcgccatgcctgagggatacgtgcaggagaggaccat
cttcttcaaggacgacgggaactacaagacacgtgctgaagtcaagtttgagggagacaccctcgtcaacagg
atcgagcttaagggaatcgatttcaaggaggacggaaTTTAAATGTGTAAGAATTTCTTATGTTA
CATTATTACATTCAACGTTTTATCTTAATTGGCTCTTCATTTGATTGAAATTTGACAATTATTTCTTGTTTTTT
TTTTTGTCACACTCTTTTTGGGTTGGGGTGGCCGACGAATTGTGGGAAGGTAGAAAGAGGGGAGGACTTTTGTT
ATACTCCATTAGTAATTACTGTTTCCGTTTCAATTTATGTGACAATATTTCCTTTTTAGTCGGTTCCAAAAGAA
AATGTCAGCATTATAAACAATTTAATTTTGAAATTACAATTTTGCCATTAATAAAATGATTTACAACCACAAAA
GTATCTATGAGCCTGTTTGGGTGGGCTTATAAGCAGCTTATTTTAAGTGGCTTATAAGTCAAAAAGTGACATTT
TTGAGAAGTTAGAAAATCCTAACTTCTCAAAAAGTAGCTTTTAAGCCACTTATGACTTATAAGTCCAAAAATTT
TTAAGTTACCAAACATATATTAATGGGTTTATAAACTTATAAAGCCACTTTTAAACTCACCCAACGGGTTCTAT
GTCTCACTTTAGACTACAAATTTTAAAAGTCTTCATTTATTTCTTAATCTCCGTGGCGAGTAAACTATAACACA
TAAAGTGAAACGGAGGGAATAAGATGGAGTCATAAACTAATCCAAATCTATACTCTCTCCGTTAATTTGTTTTT
TAGTTTGATTTGGTACATTAATAAAACAGATTTTTCGAAGGTTATAAACACAGACAGATGTTTCCCAGCGAGCT
AGCAAAATTCCAAGATTTCTGTCGAAAATTCGTGTGTTTCTAGCTAGTACTTGATGTTATCTTTAACCTTTTAG
TAATTTTTTGTCCTTTTCTTTCTATTTTTCATCTTACAATGAATTATGAGCAAGTTCCTTAAGTAGCATCACAC
GTGAGATGTTTTTTATGATATTGACTAAATCCAATCTTTACCATTCCTTAAAAACTACTATACAACACATGTTA
ATTGATACATTGCTTAACACTGAGGTTAGAAAATTTTAGAAATTAGTTGTCCAAATGCTTTGAAATTAGAAATC
TTTAATCCCTTATTTTTTTTTAAAATGTTTTTTCTCACTCCAAAGAAAGAGAAACTGACATGAAAGCTCAAAAG
ATCATGAATCTTACTAACTTTGTCCAACTAAATGTACATCAGAATGTTTCTGACATGTGAAAATGAAAGCTCTT
AATTTTCTTCTTTTATTTATTGAGGGTTTTTGCATGCTATGCATTCAATTTGAGTACTTTAAAGCACCTATAAA
CACTTACTTACACTTGCCTTGGAGTTTATGTTTTAGTGTTTTCTTCACATCTTTTTTCGTCAATTTGCAGCTAT
TGGATCCTAGGTGAGTCTAGATTTAAAttccgtcctccttgaaatcgattccctta
Agctcgatcctgttgacgagggtgtctccctcaaacttgacttcagcacgtgtcttgtagttcccgtcgtcctt
gaag
Aagatggtcctctcctgcacgtatccctcaggcatggcgctcttgaagaagtcgtgccgcttcatatgatctgg
gta
Tcttgaaaagcattgaacaccataagagaaagtagtgacaagtgttggccatggaacaggtagttttccagtag
t
Gcaaataaatttaagggtaagttttccgtatgttgcatcaccttcaccctctccactgacagaaaatttgtgcc
catt
AacatcaccatctaattcaacaagaattgggacaactccagtgaaaagttcttctcctttactGGCGCGCCCG
GGActagtccctagagtcctgtctttaatgagatatgcgagacgcctatgatcgcatgatatttgctttcaatt
ctgttgtgcac
Gttgtaaaaaacctgagcatgtgtagctcagatccttaccgccggtttcggttcattctaatgaatatatcacc
cgttactatcgta
Tttttatgaataatattctccgttcaatttactgattgtaccctactacttatatgtacaatattaaaatgaaa
acaatatattgtgctgaa
Taggtttatagcgacatctatgatagagcgccacaataacaaacaattgcgttttattattacaaatccaattt
taaaaaaagcgg
Cagaaccggtcaaacctaaaagactgattacataaatcttattcaaatttcaaaagtgccccaggggctagtat
ctacgacaca
Ccgagcggcgaactaataacgctcactgaagggaactccggttccccgccggcgcgcatgggtgagattccttg
aagttga
gtattggccgtccgctctaccgaaagttacgggcaccattcaacccggtccagcacggcggccgggtaaccgac
ttgctgccccgagaattatgcagcatttttttg
gtgtatgtgggccccaaatgaagtgcaggtcaaaccttgacagtgacgacaaatcgttgggcgggtccagggcg
aattttg
cgacaacatgtcgaggctcagcaggacctgcaggcatgcaagctt

4- Inducible promoter and hairpin GF construct in pTAdsGF
gatatcgtggatccaagcttgccacgtgccgccacgtgccgccacgtgccgccacgtgcctctagaggatccat
ctccac
tgacgtaagggatgacgcacaatcccactatccttcgcaagacccttcctctataeaaggaagttcatttcatt
tggaga
ggacacgctgggatccccaaacaatggcagatccaatgaagctactgtcttctatcgaacaagcatgcgatatt
tgccga
cttaaaaagctcaagtgctccaaagaaaaaccgaagtgcgccaagtgtctgaagaacaactgggagtgtcgcta
ctctcc
caaaaccaaaaggtctccgctgactagggcacatatgacagaagtggaatcaaggctagaaagactggaacagc
tatttc
tactgatttttcctaggtcgagcgcccccccgaccgatgtcagcctgggggaagagctccacttagacggcgag
gacgtg
gcgatggcgcatgccgacgcgctagacgatttcgatctggacatgttgggggacggggattccccgggtccggg
atttac
cccccacgactccgccccctacggcgctctggatatggccgacttcgagtttgagcagatgtttaccgatgccc
ttggaa
ttgacgagtacggtggggatccaattcagcaagccactgcaggagtctcacaagacacttcggaaaatcctaac
aaaaca
atagttcctgctgcattaccacagctcacccctaccttggtgtcactgctggaggtgattgaacccgaggtgtt
gtatgc
aggatatgatagctctgttccagattcagcatggagaattatgaccacactcaacatgttaggtgggcgtcaag
tgatty
cagcagtgaaatgggcaaaggcgataccaggcttcagaaacttacacatggatgaccaaatgaccctgctacay
tactca
tggatgtttctcatggcatttgccctgggttggagatcatacagacaatcaagcggaaacctgctctgctttgc
tcctga
tctgattattaatgagcagagaatgtctctaccctgcatgtatgaccaatgtaaacacatgctgtttgtctcct
ctgaat
tacaaagattgcaggtatcctatgaagagtatctctgtatgaaaaccttactgcttctctcctcagttcctaag
gaaggt
ctgaagagccaagagttatttgatgagattcgaatgacttatatcaaagagctaggaaaagccatcgtcaaaag
ggaagg
gaactccagtcagaactggcaacggttttaccaactgacaaagcttctggactccatgcatgaggtggttgaga
atctcc
ttacctactgcttccagacatttttggataagaccatgagtattgaattcccagagatgttagctgaaatcatc
actaat
cagataccaaaatattcaaatggaaatatcaaaaagcttctgtttcatcaaaaatgactcgacctaactgagta
agctag
cttgttcgagtattatggcattgggaaaactgtttttcttgtaccatttgttgtgcttgtaatttactgtgttt
tttatt
cggttttcgctatagaactgtgaaatggaaatggatggagaagagttaatgaatgatatggtccttttgttcat
tctcaa
attaatattatttgttttttctcttatttgttgtgtgttgaatttgaaattataagagatatgcaaacattttg
ttttga
gtaaaaatgtgtcaaatcgtggcccctaatgaccgaagttaatatgaggagtaaaacactagatcccaaacaay
cttgaa
gcttgaaactgaaggcgggaaacgacaatctgatcatgagcggagaattaagggagtcacgttatgacccccgc
cgatga
cgcgggacaagccgttttacgtttggaactgacagaaccgcaacgattgaaggagccactcagccgcgggtttc
tggagt
ttaatgagctaagcacatacgtcagaaaccattattgcgcgttcaaaagtcgcctaaggtcactatcagctagc
aaatat
ttcttgtcaaaaatgctccactgacgttccataaattcccctcggtatccaattagagtctcatattcactctc
aatcca
aataatctgcaccggatcccctagaatgtttgaacgatctgcttgactctaggggtcatcagatttcggtgacg
ggcagg
accggacggggcggcaccggcaggctgaagtccagctgccagaaacccacgtcatgccagttcccgtgcttgaa
gccggc
cgcacgcagcatgccacggggggcatatccgagcgcctcgtgcatgcgcacgctcgggtcgttgggcagcccga
tgacag
cgaccacgctcttgaagccctgtgcctccagggacttcagcaggtgggtgtagagcgtggagcccagtcccgtc
cgctgg
tggcggggggagacgtacacggttgactcggccgtccagtcgtaggcgttgcgtgccttccagggacccgcgta
ggcgat
gccggcgacctcgccgtccacctcggcgacgagccagggatagcgctcccgcagacggacgaggtcgtccgtcc
actcct
gcggttcctgcggctcggtacggaagttgaccgtgcttgtctggatgtagtggttgacgatggtgcagaccgcc
ggcaty
tccgcctcggtggcacggcggatgtcggccgggcgtcgttctgggctcatggtagatccccctcgatcgagttg
acgata
gttcaaacatttggcaataaagtttcttaagattgaatcctgttgccggtcttgcgatgattatcatataattt
ctgttg
aattacgttaagcatgtaataattaacatgtaatgcatgacgttatttatgagatgggtttttatgattagagt
cccgca
attatacatttaatacgcgatagaaaacaaaatatagcgcgcaaactaggataaattatcgcgcgcggtgtcat
ctatgt
tactagatcggggaattgatcccccctcgacagcttgcatgccggtcgactctagaggatccgggtgacagccc
tccgac
gggtgacagccctccgacgggtgacagccctccgaattctagaggatccgggtgacagccctccgacgggtgac
agccct
ccgacgggtgacagccctccgaattcgagctcggtacccggggatctgtcgacctcgatcgagatcttcgcaag
accctt
cctctatataaggaagttcatttcatttggagaggacacgctgaagctagtcgactctagcctcgagtataaga
gctcat
ttttacaacaattaccaacaacaacaaacaacaaacaacattacaattacatttacaattatccatGGCGCGCC
agtaaa
ggagaagaacttttcactggagttgtcccaattcttgttgaattagatggtgatgttaatgggcacaaattttc
tgtcag
tggagagggtgaaggtgatgcaacatacggaaaacttacccttaaatttatttgcactactggaaaactacctg
ttccat
ggccaacacttgtcactactttctcttatggtgttcaatgcttttcaagatacccagatcatatgaagcggcac
gacttc
ttcaagagcgccatgcctgagggatacgtgcaggagaggaccatcttcttcaaggacgacggqaactacaagac
acgtgc
tgaagtcaagtttgagggagacaccctcgtcaacaggatcgagcttaagggaatcgatttcaaggaggacggaa
TTTaaatGTGTAAGAATTTCTTATGTTACATTATTACATTCAACGTTTTATCTTAATTGGCTCTTCATTTGATT
GAAATTTGACAATTATTTCTTGTTTTTTTTTTTGTCACACTCTTTTTGGGTTGGGGTGGCCGACGAATTGTGGG
AAGGTAGAAAGAGGGGAGGACTTTTGTTATACTCCATTAGTAATTACTGTTTCCGTTTCAATTTATGTGACAAT
ATTTCCTTTTTAGTCGGTTCCAAAAGAAAATGTCAGCATTATAAACAATTTAATTTTGAAATTACAATTTTGCC
ATTAATAAAATGATTTACAACCACAAAAGTATCTATGAGCCTGTTTCGGTGGGCTTATAAGCAGCTTATTTTAA
GTGGCTTATAAGTCAAAAAGTGACATTTTTGAGAAGTTAGAAAATCCTAACTTCTCAAAAAGTAGCTTTTAAGC
CACTTATGACTTATAAGTCCAAAAATTTTTAAGTTACCAAACATATATTAATGGGTTTATAAGCTTATAAGCCA
CTTTTAAGCTCACCCAAACGGGTTCTATGTCTCACTTTAGACTACAAATTTTAAAAGTCTTCATTTATTTCTTA
ATCTCCGTGGCGAGTAAACTATAACACATAAAGTGAAACGGAGGGAATAAGATGGAGTCATAAACTAATCCAAA
TCTATACTCTCTCCGTTAATTTGTTTTTTAGTTTGATTTGGTACATTAATAAAACAGATTTTTCGAAGGTTATA
AACACAGACAGATGTTTCCCAGCGAGCTAGCAAAATTCCAAGATTTCTGTCGAAAATTCGTGTGTTTCTAGCTA
GTACTTGATGTTATCTTTAACCTTTTAGTAATTTTTTGTCCTTTTCTTTCTATTTTTCATCTTACAATGAATTA
TGAGCAAGTTCCTTAAGTAGCATCACACGTGAGATGTTTTTTATGATATTGACTAAATCCAATCTTTACCATTC
CTTAACTAGTAAAATACAACACATGTTAATTGATACATTGCTTAACACTGAGGTTAGAAAATTTTAGAAATTAG
TTGTCCAAATGCTTTGAAATTAGAAATCTTTAATCCCTTATTTTTTTTTAAAATGTTTTTTCTCACTCCAAAGA
AAGAGAAACTGACATGAAAGCTCAAAAGATCATGAATCTTACTAACTTTGTGGAACTAAATGTACATCAGAATG
TTTCTGACATGTGAAAATGAAA
GCTCTTAATTTTCTTCTTTTATTTATTGAGGGTTTTTGCATGCTATGCATTCAATTTGAGTACTTTAAAGCACC
TATAAACACTTACTTACACTTGCCTTGGAGTTTATGTTTTAGTGTTTTCTTCACATCTTTTTTGGTCAATTTGC
AGGTATTGGATCCTAGGTGAGTCTAGATTTAAAttccgtcctccttgaaatcgattcccttaagctcgatcctg
ttgacgagggtgtctccc
tcaaacttgacttcagcacgtgtcttgtagttcccgtcgtccttgaagaagatggtcctctcctgcacgtatcc
ctcagg
catggcgctcttgaagaagtcgtgccgcttcatatgatctgggtatcttgaaaagcattgaacaccataagaga
aagtag
tgacaagtgttggccatggaacaggtagttttccagtagtgcaaataaatttaagggtaagttttccgtatgtt
gcatca
ccttcaccctctccactgacagaaaatttgtgcccattaacatcaccatctaattcaacaagaattgggacaac
tccagt
gaaaagttcttctcctttactGGCGCGCCCGGATTAACTAGTCGATCCGTCGACACAAAAAGCCTATACTGTAC
TTAACTTGATTGCATAATTACTTGATCATAGACTCATAGTAAACTTGATTACACAGATAAGTGAAGAAACAAAC
CAATTCAAGACATAACCAAAGAGAGGTGAAAGACTGTTTTATATGTCTAACATTGCACCTTAATATCACACTGT
TAGTTCCTTTCTTACTTAAATTCAACCCATTAAAGTAAAAACAACAGATAATAATAATTTGAGAATGAACAAAA
GGACCATATCATTTATTAACTCTTATCCATCCATTTGCATTTTGATGTCCGAAAACAAAAACTGAAAGAACACA
GTAAATTACAAGCAGAACAAATGATAGAAGAAAACAGCTTTTCCAATGCCATAATACTCAAACTTAGTAGGATT
CTGGTGTGTGGGCAATGAAACTGATGCATTGAACTTGACGAACGTTGTCGAAACCGATGATACGGACGAAAGCT
TCGAGAATTC

1. A method of silencing a target gene in an organism, which method comprises the steps of:
(a) providing a recombinant DNA construct including an expression cassette comprising:
(i) a promoter, operably linked to,
(ii) a chimeric nucleotide sequence encoding all or part of the target gene and a transgene,
(b) transforming the organism with said DNA construct such that the expression cassette is inserted into the genome, and
(c) initiating post transcriptional gene silencing (PTGS) of said transgene in said organism,
whereby initiation of PTGS of the transgene causes silencing of the target gene in the organism.

2. A method as claimed in claim 1 wherein the target gene is an endogenous gene and whereby PTGS of the transgene spreads in trans to silence said target gene.

3. A method as claimed in claim 1 wherein the chimeric nucleotide sequence includes at least the initiating ATG codon of the target gene.

4. A method as claimed in claim 1 wherein the sequence encoding all or part of the target gene is inserted within the sequence encoding all or part of the transgene.

5. A method as claimed in claim 1 wherein PTGS of the transgene in the organism is initiated at step (c) by introducing into the organism a second nucleic acid construct which includes sequence corresponding to the transgene sequence.

6. A method as claimed in claim 5 wherein the constructs of step (a) and step (c) each comprise an inducible promoter.

7. A method as claimed claim 1 wherein PTGS of the transgene in the organism is initiated at step (c) by introducing into the organism a virus, or sequence derived therefrom, carrying all or part of the transgene sequence.

8. A method as claimed in claim 1 wherein PTGS of the transgene in the organism is initiated at step (c) by introducing into the organism a hairpin construct carrying an inverted repeat of all or part of the transgene sequence.

9. A method as claimed in claim 1 wherein PTGS of the transgene is extant in the organism prior to transformation with the recombinant DNA construct.

10. A method as claimed in claim 9, which method comprises the steps of:
(a) providing or selecting an organism which has been transformed with a transgene which has been subject to PTGS,
(b) providing a recombinant DNA construct including an expression cassette comprising:
(i) a promoter, operably linked to,
(ii) a chimeric nucleotide sequence encoding all or part of the target gene and a transgene,
(c) transforming the organism with said DNA construct such that the expression cassette is inserted into the genome.

11. A method as claimed in preceding claim 1 wherein the organism is a plant.

12. A method as claimed in claim 11 wherein the recombinant DNA construct including an expression cassette comprising:
(i) a promoter, operably linked to,
(ii) a chimeric nucleotide sequence encoding all or part of the target gene and a transgene,
further includes border sequences situated around the expression cassette capable of being inserted into a plant genome.

13. A recombinant DNA construct including an expression cassette comprising:
(i) a promoter, operably linked to
(ii) a chimeric nucleotide sequence encoding all or part of a target gene endogenous to a plant, and a transgene,
the construct further comprising
(iii) border sequences situated around said expression cassette, capable of being inserted into a plant genome.

14. A construct as claimed in claim 13 wherein the chimeric nucleotide sequence includes at least the initiating ATG codon of the target gene.

15. A construct as claimed in claim 13 wherein the sequence encoding all or part of the target gene is inserted within the sequence encoding all or part of the transgene.

16. A construct as claimed in claim 13 wherein the transgene is GFP or GUS.

17. A construct as claimed in claim 13 wherein the target gene is associated with a trait in the plant.

18. A composition comprising a plurality of recombinant DNA constructs wherein said DNA constructs include an expression cassette comprising:
(i) a promoter, operably linked to
(ii) a chimeric nucleotide sequence encoding all or part of a target gene endogenous to a plant, and a transgene, said target gene sequence optionally being inserted within the sequence encoding part or all of the transgene and optionally being associated with a trait in the plant,
the construct further comprising
(iii) border sequences situated around said expression cassette, capable of being inserted into a plant genome, said chimeric nucleotide sequence optionally including the initiating ATG codon of the target gene, wherein each construct includes a different target gene from the same plant.

19. A composition as claimed in claim 18 wherein each construct includes a target gene from the same cDNA library.

20. A method which comprises the step of introducing at least one DNA construct including an expression cassette comprising:
(i) a promoter, operably linked to
(ii) a chimeric nucleotide sequence encoding all or part of a target gene endogenous to a plant, and a transgene, said target gene sequence optionally being inserted within the sequence encoding part or all of the transgene and optionally being associated with a trait in the plant,
the construct further comprising
(iii) border sequences situated around said expression cassette, capable of being inserted into a plant genome, said chimeric nucleotide sequence optionally including the initiating ATG codon of the target gene into a plant cell, and causing or allowing recombination between the construct and the plant cell genome such as to introduce the expression cassette into the genome.

21. A method as claimed in claim 20 wherein said plant cell is a recombinant plant cell transformed with a second DNA construct capable of triggering PTGS of the transgene, said second construct optionally including an inducible promoter.

22. A method as claimed in claim 21 wherein said second DNA construct capable of triggering PTGS of the transgene comprises an expression cassette comprising (i) a promoter, operably linked to (ii) DNA for transcription in a plant cell of an RNA molecule that includes (I) plant virus sequences that confer on the RNA molecule the ability to replicate in the cytoplasm of the plant cell following transcription (II) a targeting sequence corresponding to the transgene.

23. A method as claimed in claim 21 wherein said second DNA construct capable of triggering PTGS of the transgene comprises a hairpin construct carrying an inverted repeat of all or part of the transgene sequence.

24. A method as claimed in claim 21 wherein the triggering of the PTGS of the transgene is controlled by an inducible promoter.

25. A plant cell obtainable by the method of claim 20.

26. A method for producing a transgenic plant, which method comprises the steps of:
(a) performing a method as claimed in claim 25,
(b) regenerating a plant from the transformed plant cell.

27. A transgenic plant which is obtainable by the method of claim 26, or which is a clone, or selfed or hybrid progeny or other descendant of said transgenic plant.

28. A method of silencing a target gene in a plant, which method comprises the steps of:
(a) providing a plant as claimed in claim 27,
(b) initiating PTGS of said transgene in the plant.

29. A method as claimed in claim 28 wherein a plurality of plants are provided each containing a different target gene, and PTGS is initiated in each.

30. A method a silencing a target gene in a plant, which method comprises the steps of:
(a) providing a first DNA construct which includes an expression cassette comprising:
(i) a promoter, operably linked to
(ii) a chimeric nucleotide sequence encoding all or part of a target gene endogenous to a plant, and a transgene, said target gene sequence optionally being inserted within the sequence encoding part or all of the transgene and optionally being associated with a trait in the plant,
the construct further comprising
(iii) border sequences situated around said expression cassette, capable of being inserted into a plant genome, said chimeric nucleotide sequence optionally including the initiating ATG codon of the target gene,
(b) providing a second DNA construct including an expression cassette comprising (i) a promoter, operably linked to (ii) DNA for transcription in a plant cell of an RNA molecule that includes (I) plant virus sequences that confer on the RNA molecule the ability to replicate in the cytoplasm of a plant cell following transcription (II) a targeting sequence corresponding to the transgene,
(c) transforming the organism with said DNA constructs such that the expression cassettes are inserted into the genome, and
optionally
(d) causing or permitting transcription of the expression cassettes such as to cause silencing of the target gene in an organism.

31. A method as claimed in claim 28 wherein the, or each plant phenotype is observed after the target gene is silenced.

32. A method as claimed in claim 31 wherein the, or each observation contrasted with a plant wherein the target gene is being expressed.

33. A method of characterizing a target gene comprising the steps of:
(a) silencing the target gene in a part or at a certain development stage of the plant by use of a method of claim 28,
(b) observing the phenotype of the part of the plant in which, or when, the target gene has been silenced.

34. A method as claimed in claim 28 further comprising the step of amplifying the silenced target gene sequence from the expression cassette in the transformed plant.

35. A method as claimed in claim 2 wherein the chimeric nucleotide sequence includes at least the initiating ATG codon of the target gene.

36. A construct as claimed in claim 14 wherein the sequence encoding all or part of the target gene is inserted within the sequence encoding all or part of the trans gene.

37. A plant cell obtainable by the method of claim 21.

38. A plant cell obtainable by the method of claim 22.

39. A plant cell obtainable by the method of claim 23.

40. A method for producing a transgenic plant, which method comprises the steps of:
(a) performing a method as claimed in claim 21,
(b) regenerating a plant from the transformed plant cell.

41. A method for producing a transgenic plant, which hod comprises the steps of:
(a) performing a method as claimed in claim 22,
(b) regenerating a plant from the transformed plant cell.

42. A method for producing a transgenic plant, which method comprises the steps of:
(a) performing a method as claimed in claim 23,
(b) regenerating a plant from the transformed plant cell.

43. A method as claimed in claim 29 wherein the, or each plant phenotype is observed after the target gene is silenced.

44. A method as claimed in claim 30 wherein the, or each plant phenotype is observed after the target gene is silenced.

45. A method of characterizing a target gene comprising the steps of:
(a) silencing the target gene in a part or at a certain development stage of the plant by use of a method of claim 30
(b) observing the phenotype of the part of the plant in which, or when, the target gene has been silenced.

46. A method of characterizing a target gene comprising steps of:
(a) silencing the target gene in a part or at a certain development stage of the plant by use of a method of claim 31
(b) observing the phenotype of the part of the plant in which, or when, the target gene has been silenced.

47. A method of characterizing a target gene comprising the steps of:
(a) silencing the target gene in a part or at a certain development stage of the plant by use of a method of claim 32
(b) observing the phenotype of the part of the plant in which, or when, the target gene has been silenced.


SEQUENCE LISTING (30 sequences)

SEQ ID NO: 1 - DNA, 9487 bp, Artificial sequence
  Synthetic sequence
tcttggcaggatatattgtggtgtaacgttatcagcttgcatgccggtcgatctagtaacatagatgacaccgcgcgcgataatttatcctagtttgcgcgctatattttgttttctatcgcgtattaaatgtataattgcgggactctaatcaaaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccctgggtcacgacgagatcctcgccgtcgggcatccgcgccttgagcctggcgaacagttcggctggcgcgagcccctgatgctcttcgtccagatcatcctgatcgacaagaccggcttccatccgagtacgtcctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtatcgataacattaacgtttacaatttcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagctttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaatttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggatcatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgccaagtgggaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaagctttctagaggatcccccggggccttggcgcgccttcactctcttctccttcctcaaaaccttcctcctcccccatttgcttcaggccaggtaaattgtttggaagcaagttaaatgcaggaatccaaataaggccaaagaagaacaggtctcgttaccatgtttcggttatgaatgtagccactgaaatcaactctactgaacaagtagtagggaagtttgattcaaagaagagtgcgagaccggtttatccatttgcagctatagtagggcaagatgagatgaagttatgtcttttgttgaatgttattgatccaaagattggtggtgttatgattatgggagatagaggaactggaaaatctacaactgttagatcattagttgatctgttacctgagattaatgtagttgcaggtgacccgtataactcggatccgatagatcctgagtttatgggtgttgaagtaagagagagagttgagaaaggagagcaagttcctgttattgcgactaagattaatatggttgatcttcctttgggtgcaacagaagatagagtttgtggaaccatcgatatcgaaaaggctttgacagaaggtgtaaaagcctttgagcctggtttgttggctaaagctaatagagggattctttatgttgatgaagttaatctcttggatgatcatttggttgatgttcttttggattcagctgcttctggttggaatacggttgagagagaagggatttcgatttctcacccggcgaggtttatcttgatcggttcaggaaatccggaagaaggagagcttaggccacagcttcttgatcggtttggtatgcatgcacaagtagggacggttagagatgctgatttacgggtcaagattgttgaagagagagctcgtttcgatagtaacccaaaggatttccgtgacacttacaaaaccgagcaggacaagcttcaagaccagattaattaaggggcgaatttcgaccgccgataagcttgatagggccattgccgatctcaagccactctccgttgaacggttaagtttccattgatactcgaaagatgtcagcaccagctagcacaacacagcccatagggtcaactacctcaactaccacaaaaactgcaggcgcaactcctgccacagcttcaggcctgttcaccatcccggatggggatttctttagtacagcccgtgccatagtagccagcaatgctgtcgcaacaaatgaggacctcagcaagattgaggctatttggaaggacatgaaggtgcccacagacactatggcacaggctgcttgggacttagtcagacactgtgctgatgtaggatcatccgctcaaacagaaatgatagatacaggtccctattccaacggcatcagcagagctagactggcagcagcaattaaagaggtgtgcacacttaggcaattttgcatgaagtatgctccagtggtatggaactggatgttaactaacaacagtccacctgctaactggcaagcacaaggtttcaagcctgagcacaaattcgctgcattcgacttcttcaatggagtcaccaacccagctgccatcatgcccaaagaggggctcatccggccaccgtctgaagctgaaatgaatgctgcccaaactgctgcctttgtgaagattacaaaggccagggcacaatccaacgactttgccagcctagatgcagctgtcactcgaaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggagcatgcgatccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaaggccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagaga

SEQ ID NO: 2 - DNA, 4162 bp, Artificial sequence
  Synthetic sequence
ggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagctttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaatttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggatcatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgccaagtgggaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaagctttctagaggatcccccggggcgaattccttggcgcgccttcactctcttctccttcctcaaaaccttcctcctcccccatttgcttcaggccaggtaaattgtttggaagcaagttaaatgcaggaatccaaataaggccaaagaagaacaggtctcgttaccatgtttcggttatgaatgtagccactgaaatcaactctactgaacaagtagtagggaagtttgattcaaagaagagtgcgagaccggtttatccatttgcagctatagtagggcaagatgagatgaagttatgtcttttgttgaatgttattgatccaaagattggtggtgttatgattatgggagatagaggaactggaaaatctacaactgttagatcattagttgatctgttacctgagattaatgtagttgcaggtgacccgtataactcggatccgatagatcctgagtttatgggtgttgaagtaagagagagagttgagaaaggagagcaagttcctgttattgcgactaagattaatatggttgatcttcctttgggtgcaacagaagatagagtttgtggaaccatcgatatcgaaaaggctttgacagaaggtgtaaaagcctttgagcctggtttgttggctaaagctaatagagggattctttatgttgatgaagttaatctcttggatgatcatttggttgatgttcttttggattcagctgcttctggttggaatacggttgagagagaagggatttcgatttctcacccggcgaggtttatcttgatcggttcaggaaatccggaagaaggagagcttaggccacagcttcttgatcggtttggtatgcatgcacaagtagggacggttagagatgctgatttacgggtcaagattgttgaagagagagctcgtttcgatagtaacccaaaggatttccgtgacacttacaaaaccgagcaggacaagcttcaagaccagattaattaaggggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggagcatgcgatccagcttttgttc

SEQ ID NO: 3 - DNA, 3435 bp, Artificial sequence
  pGF/FG. Showed from promoter to terminator.
tactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatccatggcgcgccagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaatttaaatgtgtaagaatttcttatgttacattattacattcaacgttttatcttaattggctcttcatttgattgaaatttgacaattatttcttgtttttttttttgtcacactctttttgggttggggtggccgacgaattgtgggaaggtagaaagaggggaggacttttgttatactccattagtaattactgtttccgtttcaatttatgtgacaatatttcctttttagtcggttccaaaagaaaatgtcagcattataaacaatttaattttgaaattacaattttgccattaataaaatgatttacaaccacaaaagtatctatgagcctgtttgggtgggcttataagcagcttattttaagtggcttataagtcaaaaagtgacatttttgagaagttagaaaatcctaacttctcaaaaagtagcttttaagccacttatgacttataagtccaaaaatttttaagttaccaaacatatattaatgggtttataagcttataagccacttttaagctcacccaaacgggttctatgtctcactttagactacaaattttaaaagtcttcatttatttcttaatctccgtggcgagtaaactataacacataaagtgaaacggagggaataagatggagtcataaactaatccaaatctatactctctccgttaatttgttttttagtttgatttggtacattaataaaacagatttttcgaaggttataaacacagacagatgtttcccagcgagctagcaaaattccaagatttctgtcgaaaattcgtgtgtttctagctagtacttgatgttatctttaaccttttagtaattttttgtccttttctttctatttttcatcttacaatgaattatgagcaagttccttaagtagcatcacacgtgagatgttttttatgatattgactaaatccaatctttaccattccttaactagtaaaatacaacacatgttaattgatacattgcttaacactgaggttagaaaattttagaaattagttgtccaaatgctttgaaattagaaatctttaatcccttatttttttttaaaatgttttttctcactccaaagaaagagaaactgacatgaaagctcaaaagatcatgaatcttactaactttgtggaactaaatgtacatcagaatgtttctgacatgtgaaaatgaaagctcttaattttcttcttttatttattgagggtttttgcatgctatgcattcaatttgagtactttaaagcacctataaacacttacttacacttgccttggagtttatgttttagtgttttcttcacatcttttttggtcaatttgcaggtattggatcctaggtgagtctagatttaaattccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactggcgcgcccgggactagtccctagagtcctgtctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctt

SEQ ID NO: 4 - DNA, 6128 bp, Artificial sequence
  Inducible promoter and hairpin GF construct in pTAdsGF
gatatcgtggatccaagcttgccacgtgccgccacgtgccgccacgtgccgccacgtgcctctagaggatccatctccactgacgtaagggatgacgcacaatcccactatccttcgcaagacccttcctctatataaggaagttcatttcatttggagaggacacgctgggatccccaaacaatggcagatccaatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctaggtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccaattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagcggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatgactcgacctaactgagtaagctagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactagatcccaaacaagcttgaagcttgaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatcccctagaatgtttgaacgatctgcttgactctaggggtcatcagatttcggtgacgggcaggaccggacggggcggcaccggcaggctgaagtccagctgccagaaacccacgtcatgccagttcccgtgcttgaagccggccgcccgcagcatgccacggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggttgactcggccgtccagtcgtaggcgttgcgtgccttccagggacccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctggatgtagtggttgacgatggtgcagaccgccggcatgtccgcctcggtggcacggcggatgtcggccgggcgtcgttctgggctcatggtagatccccctcgatcgagttgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggaattgatcccccctcgacagcttgcatgccggtcgactctagaggatccgggtgacagccctccgacgggtgacagccctccgacgggtgacagccctccgaattctagaggatccgggtgacagccctccgacgggtgacagccctccgacgggtgacagccctccgaattcgagctcggtacccggggatctgtcgacctcgatcgagatcttcgcaagacccttcctctatataaggaagttcatttcatttggagaggacacgctgaagctagtcgactctagcctcgagtataagagctcatttttacaacaattaccaacaacaacaaacaacaaacaacattacaattacatttacaattatccatggcgcgccagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaatttaaatgtgtaagaatttcttatgttacattattacattcaacgttttatcttaattggctcttcatttgattgaaatttgacaattatttcttgtttttttttttgtcacactctttttgggttggggtggccgacgaattgtgggaaggtagaaagaggggaggacttttgttatactccattagtaattactgtttccgtttcaatttatgtgacaatatttcctttttagtcggttccaaaagaaaatgtcagcattataaacaatttaattttgaaattacaattttgccattaataaaatgatttacaaccacaaaagtatctatgagcctgtttgggtgggcttataagcagcttattttaagtggcttataagtcaaaaagtgacatttttgagaagttagaaaatcctaacttctcaaaaagtagcttttaagccacttatgacttataagtccaaaaatttttaagttaccaaacatatattaatgggtttataagcttataagccacttttaagctcacccaaacgggttctatgtctcactttagactacaaattttaaaagtcttcatttatttcttaatctccgtggcgagtaaactataacacataaagtgaaacggagggaataagatggagtcataaactaatccaaatctatactctctccgttaatttgttttttagtttgatttggtacattaataaaacagatttttcgaaggttataaacacagacagatgtttcccagcgagctagcaaaattccaagatttctgtcgaaaattcgtgtgtttctagctagtacttgatgttatctttaaccttttagtaattttttgtccttttctttctatttttcatcttacaatgaattatgagcaagttccttaagtagcatcacacgtgagatgttttttatgatattgactaaatccaatctttaccattccttaactagtaaaatacaacacatgttaattgatacattgcttaacactgaggttagaaaattttagaaattagttgtccaaatgctttgaaattagaaatctttaatcccttatttttttttaaaatgttttttctcactccaaagaaagagaaactgacatgaaagctcaaaagatcatgaatcttactaactttgtggaactaaatgtacatcagaatgtttctgacatgtgaaaatgaaagctcttaattttcttcttttatttattgagggtttttgcatgctatgcattcaatttgagtactttaaagcacctataaacacttacttacacttgccttggagtttatgttttagtgttttcttcacatcttttttggtcaatttgcaggtattggatcctaggtgagtctagatttaaattccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactggcgcgcccggattaactagtcgatccgtcgacacaaaaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagcttcgagaattc

SEQ ID NO: 5 - DNA, 24 bp, Artificial sequence
  Oligonucleotide
agtaaaggagaagaacttttcact

SEQ ID NO: 6 - DNA, 21 bp, Artificial sequence
  Oligonucleotide
ttccgtcctccttgaaatcga

SEQ ID NO: 7 - DNA, 20 bp, Artificial sequence
  Oligonucleotide
aacatcctcggccacaagtt

SEQ ID NO: 8 - DNA, 22 bp, Artificial sequence
  Oligonucleotide
caggtaatggttgtctggtaaa

SEQ ID NO: 9 - DNA, 21 bp, Artificial sequence
  Oligonucleotide
cgttcaaacatttggcaataa

SEQ ID NO: 10 - DNA, 21 bp, Artificial sequence
  Oligonucleotide
ctctaatcataaaaacccatc

SEQ ID NO: 11 - DNA, 21 bp, Artificial sequence
  Oligonucleotide
gagctcttagagttcgtcatg

SEQ ID NO: 12 - DNA, 29 bp, Artificial sequence
  Oligonucleotide
cagggatccggcactcaactttataaacc

SEQ ID NO: 13 - DNA, 30 bp, Artificial sequence
  Oligonucleotide
cagtctagatcccttcagttttctgtcaaa

SEQ ID NO: 14 - DNA, 22 bp, Artificial sequence
  Oligonucleotide
aatgaggatggaagtgtcaaat

SEQ ID NO: 15 - DNA, 22 bp, Artificial sequence
  Oligonucleotide
cagctcgatcttttttattcgt

SEQ ID NO: 16 - DNA, 29 bp, Artificial sequence
  Oligonucleotide
cagggatcctggcttcctcagttctttcc

SEQ ID NO: 17 - DNA, 26 bp, Artificial sequence
  Oligonucleotide
cagtctngacacttgacgcacgggtc

SEQ ID NO: 18 - DNA, 21 bp, Artificial sequence
  Oligonucleotide
gaaaaatggatgggttccttg

SEQ ID NO: 19 - DNA, 21 bp, Artificial sequence
  Oligonucleotide
aaaaggtactcaacttcacta

SEQ ID NO: 20 - DNA, 24 bp, Artificial sequence
  Oligonucleotide
atacgtgcaggagaggaccattct

SEQ ID NO: 21 - DNA, 22 bp, Artificial sequence
  Oligonucleotide
acgagggtgtctccctcaaact

SEQ ID NO: 22 - DNA, 28 bp, Artificial sequence
  Oligonucleotide
acgacgggaactacaagacacgtgctga

SEQ ID NO: 23 - DNA, 24 bp, Artificial sequence
  Primer
ttatgttacgtcctgtagaaaccc

SEQ ID NO: 24 - DNA, 22 bp, Artificial sequence
  Primer
tcattgtttgcctccctgctgc

SEQ ID NO: 25 - DNA, 31 bp, Artificial sequence
  Primer
ccttggcgcgccttcactctcttctccttcc

SEQ ID NO: 26 - DNA, 32 bp, Artificial sequence
  Primer
ccccttaattaatctggtcttgaagcttgtcc

SEQ ID NO: 27 - DNA, 32 bp, Artificial sequence
  Primer
ccatatgtttaaacccctactccaaaaatgtc

SEQ ID NO: 28 - DNA, 32 bp, Artificial sequence
  Primer
cccgtagtttaaacgtcgaggatatcgcatgc

SEQ ID NO: 29 - DNA, 35 bp, Artificial sequence
  Primer
aattcccgggcgcgccatgaaaggagaagaacttt

SEQ ID NO: 30 - DNA, 34 bp, Artificial sequence
  Primer
atattctagatttaaattccgtcctccttgaaat